(19) United States
(12) Patent Application Publication
(10) Pub. No.: US 2024/0368219 A1
(43) Pub. Date: Nov. 7, 2024

(54) ACTIVATION OF E6AP/UBE3A-MEDIATED PROTEIN UBIQUITINATION AND DEGRADATION PATHWAYS BY A CYCLIC GAMMA-AA PEPTIDE

(71) Applicants: University of South Florida, Tampa, FL (US); Georgia State University Research Foundation, Inc., Atlanta, GA (US)

(72) Inventors: Jianfeng Cai, Tampa, FL (US); Bo Huang, Tampa, FL (US); Jun Yin, Atlanta, GA (US); Li Zhou, Atlanta, GA (US); Ruochuan Liu, Atlanta, GA (US)

(21) Appl. No.: 18/776,994

(22) Filed: Jul. 18, 2024

Related U.S. Application Data
(63) Continuation of application No. PCT/US2023/011057, filed on Jan. 18, 2023.
(60) Provisional application No. 63/266,907, filed on Jan. 18, 2022.

(51) Int. Cl. C07K 5/10; A61K 38/00

(52) U.S. Cl. CPC C07K 5/10; A61K 38/00

(57) ABSTRACT

Novel cyclic γ-AA peptides and methods of use thereof in modulating E3 activity are presented. Each novel compound is comprised of four γ-AA building blocks. It was found that in particular, compound P6 activated E6AP to stimulate self-ubiquitination of E6AP and E6AP-catalyzed substrate ubiquitination as well as enhance the ubiquitination of E6AP substrates in the cell and accelerate their degradation.

### CROSS REFERENCE TO RELATED APPLICATIONS

This application is a continuation of and claims priority to International Patent Application Serial No. PCT/US2023/011057, entitled “Activation of E6AP/UBE3A-Mediated Protein Ubiquitination and Degradation Pathways by a Cyclic γ-AA Peptide”, filed on Jan. 18, 2023, which claims priority to U.S. Provisional Patent Application Ser. No. 63/266,907, entitled “Activation of E6AP/UBE3A-Mediated Protein Ubiquitination and Degradation Pathways by a Cyclic γ-AA Peptide”, filed Jan. 18, 2022, the contents of each of which are hereby incorporated by reference into this disclosure.

### GOVERNMENT SUPPORT

This invention was made with Government support under Grant No. R01 GM104498 awarded by the National Institutes of Health (NIH) and Grant No. 2109051 awarded by the National Science Foundation (NSF). The Government has certain rights in the invention.

### FIELD OF INVENTION

This invention relates to compounds and methods of activating E3. Specifically, the invention provides novel cyclic γ-AA peptide compounds and methods activating E6AP protein ubiquitination and degradation of protein substrates.

### BACKGROUND OF THE INVENTION

E3 ubiquitin (UB) ligases are promising drug discovery targets due to their essential regulatory roles in diverse cellular processes such as protein degradation, gene activation, DNA repair, autophagy and cell cycle and differentiation 1-4. Recent work on repurposing E3s for induced protein degradation further fuels the effort for designing and screening E3 ligands for the assembly of bifunctional proteolysis-targeting chimeras (PROTAC) or monovalent molecular glues to control protein stability in the cell⁵⁻⁶. E6 associated protein (E6AP) has been a prototypical E3 for probing the catalytic mechanism of the UB transfer reaction and the roles of E3s in cell regulation⁷⁻¹¹. (FIG. 1). E6AP was identified for its association with the E6 protein of the human papillomavirus (HPV) that would stir E6AP to ubiquitinate p53 for its degradation by the proteasome. This would allow the virus to subvert the antiviral response of the host cells and promote viral infection that eventually leads to tumorigenesis¹²⁻¹⁴. For counteracting E6AP that coalesce with HPV E6 to manifest its oncogenic activity, efforts have been devoted to the screening of cyclic peptides that would bind to E6AP and inhibit p53 ubiquitination¹⁵ and small molecules and peptide ligands that would bind to E6 to prevent the formation of the E6-E6AP complex¹⁶⁻¹⁹.

On the other hand, the deletion and mutation of UBE3A, the gene encoding E6AP, is implicated in neurodevelopmental disorders such as Angelman syndrome²⁰⁻²¹. A variety of approaches have been developed to replenish E6AP activity in Angelman patients, including the use of a topoisomerase inhibitor, antisense oligonucleotides, and Cas9-mediated gene editing to activate the expression of UBE3A gene²²⁻²⁴. Furthermore, a recent screen yielded small molecules that can stimulate the UB ligase activity of E6AP as demonstrated by in vitro ubiquitination assays²⁵.

Identification of activators of a specific E3 to stimulate the ubiquitination and degradation of the native E3 targets may be a viable alternative strategy to PROTACs and molecular glues. It is well recognized that targeted protein degradation is an emerging field in chemical biology and therapeutic development. So far, PROTACs and molecular glues have been the focus of study to induce the degradation of target proteins in the cell. Both strategies require the development of bi-functional ligands binding simultaneously with E3 ubiquitin ligases and the target proteins. To induce the degradation of new targets by PROTACs or molecular glues, one has to acquire affinity ligands with the target proteins through either rational design or library screening.

In contrast, an E3 activator can be a mono-functional ligand but stimulate degradation of multiple protein substrates, which can achieve a synergistic effect in treating a variety of diseases. Such an activator may also be used to restore the function of an E3 in cell or tissues when the native activity of an E3 is suppressed by certain diseases.

What is needed is an E3 activator that can stimulate the ubiquitin ligase activity of E6AP to ubiquitinate its substrate proteins and thus restore the function of E3 when such function is suppressed by a disease to thus treat the disease.

### SUMMARY OF INVENTION

Manipulating the activities of E3 ubiquitin ligases with chemical ligands holds promise for correcting E3 malfunctions and repurposing the E3s for induced protein degradation in the cell. The inventors have developed an alternative strategy to PROTACs and molecular glues to induce protein degradation by constructing and screening a γ-AA peptide library for cyclic peptidomimetics binding to the HECT domains of E6AP, an E3 ubiquitinating p53 coerced by the human papillomavirus and regulating pathways implicated in neurodevelopmental disorders such as Angelman syndrome. The inventors found a γ-AA peptide P6, discovered from the affinity-based screen with the E6AP HECT domain, can significantly stimulate the ubiquitin ligase activity of E6AP to ubiquitinate its substrate proteins UbxD8, HHR23A, and β-catenin in reconstituted reactions and HEK293 cells. Furthermore, P6 can accelerate the degradation of E6AP substrates in the cell by enhancing the catalytic activities of E6AP. The work demonstrates the feasibility of using synthetic ligands to stimulate E3 activities in the cell. The E3 stimulators can be developed alongside E3 inhibitors and substrate recruiters such as PROTAC and molecular glues to leverage the full potential of protein ubiquitination pathways for drug development.

In an embodiment, novel compounds are presented comprising:

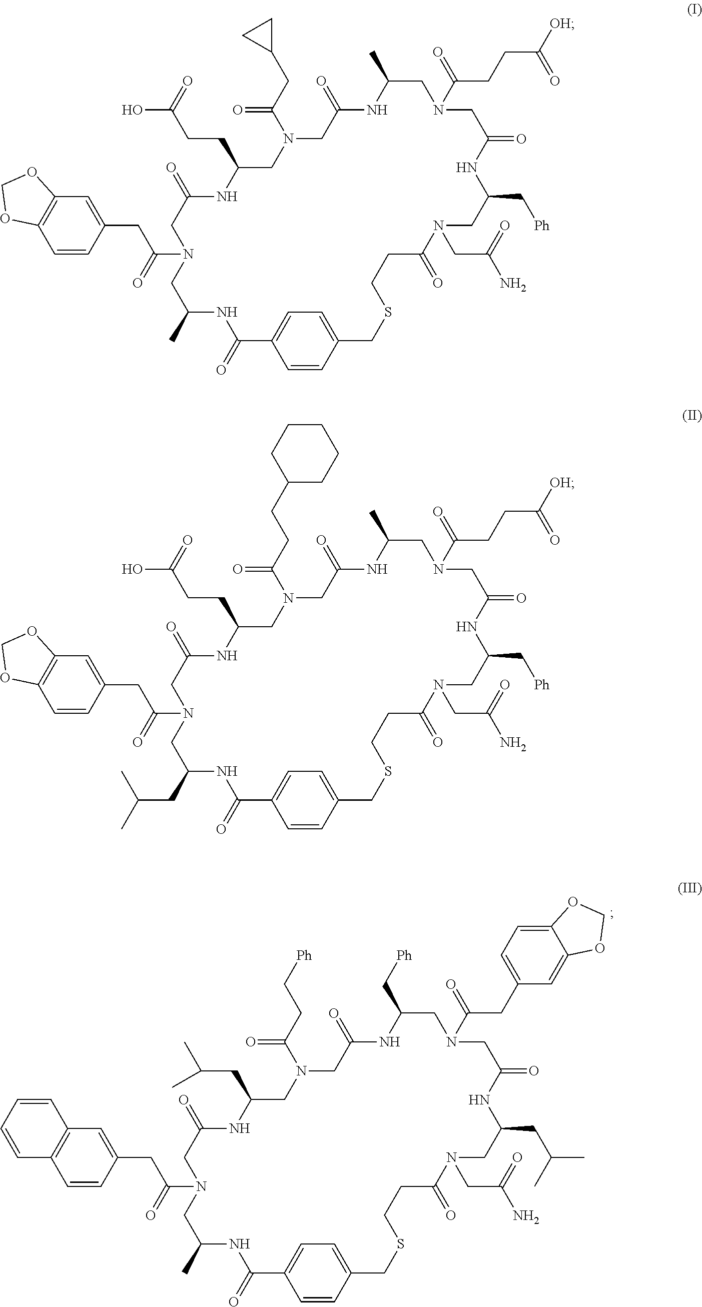

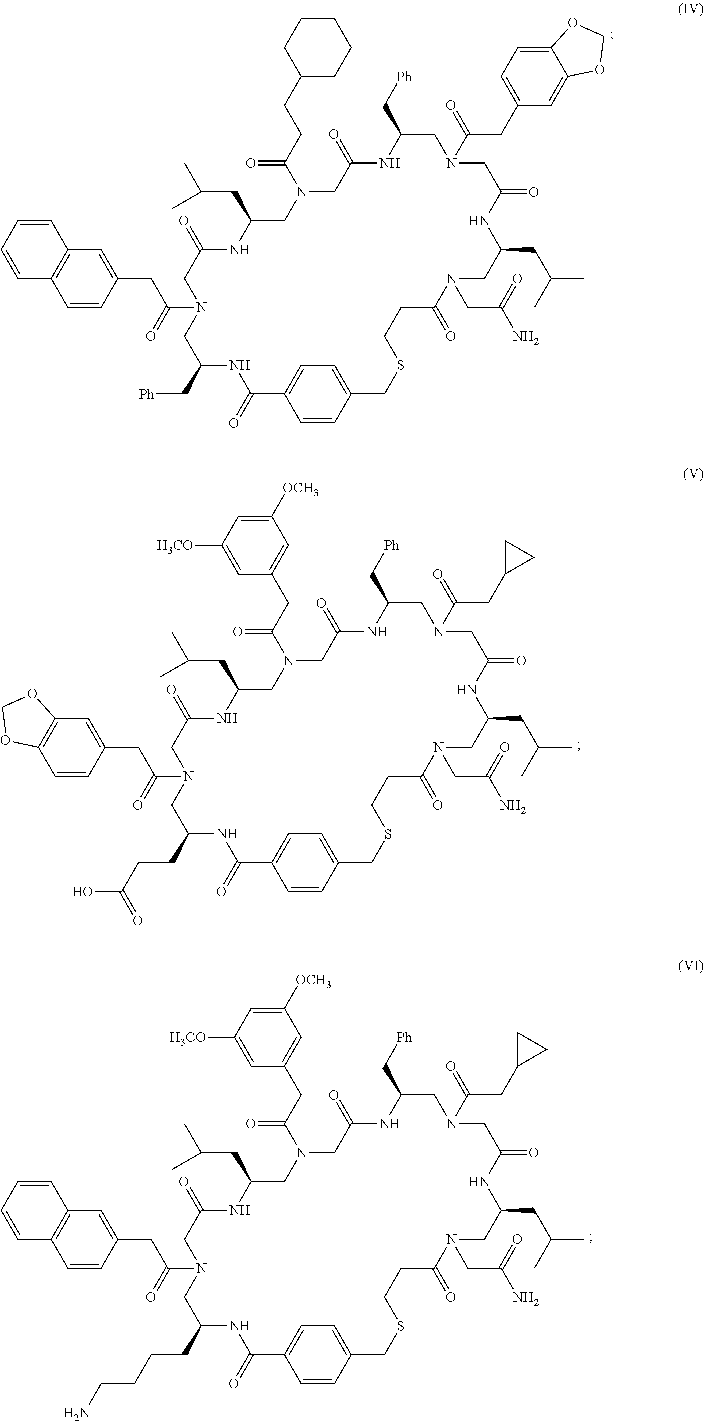

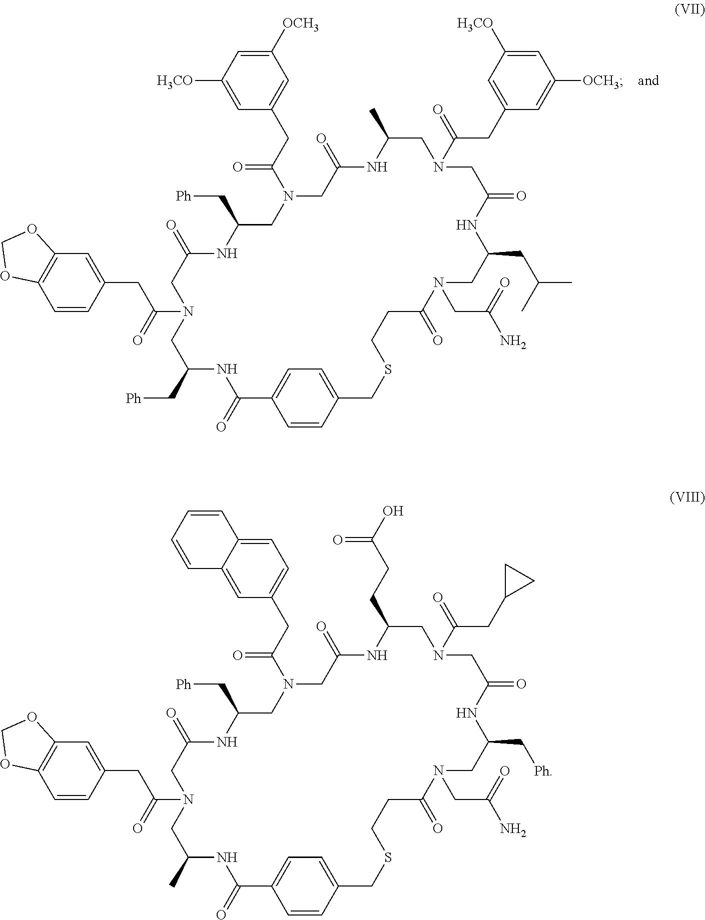 [SMILES: COc1cc(CC(=O)N2CC(=O)N[C@@H](C)CN(C(=O)Cc3cc(OC)cc(OC)c3)CC(=O)N[C@@H](CC(C)C)CN(CC(N)=O)C(=O)CCSCc3ccc(cc3)C(=O)N[C@@H](Cc3ccccc3)CN(C(=O)Cc3ccc4c(c3)OCO4)CC(=O)N[C@@H](Cc3ccccc3)C2)cc(OC)c1 ; "and" ; C[C@H]1CN(C(=O)Cc2ccc3c(c2)OCO3)CC(=O)N[C@@H](Cc2ccccc2)CN(C(=O)Cc2ccc3ccccc3c2)CC(=O)N[C@@H](CCC(=O)O)CN(C(=O)CC2CC2)CC(=O)N[C@@H](Cc2ccccc2)CN(CC(N)=O)C(=O)CCSCc2ccc(cc2)C(=O)N1]

In a further embodiment, a method of stimulating E3 ubiquitin ligase activity in a cell is presented comprising contacting the cell with a therapeutically effective amount of at least one 7-substituted-N-acylated-N-aminoethyl amino acids (γ-AA peptide) compound wherein the γ-AA peptide stimulates the E3 ubiquitin ligase activity in the cell. The E3 ubiquitin ligase may be E6 associated protein (E6AP).

The γ-AA peptide may be cyclic and formed from at least one γ-AA building block. The at least one γ-AA building block may be comprised of Formula (IX):

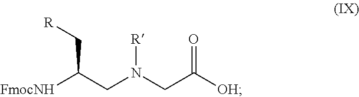

wherein R is selected from the group consisting of H,

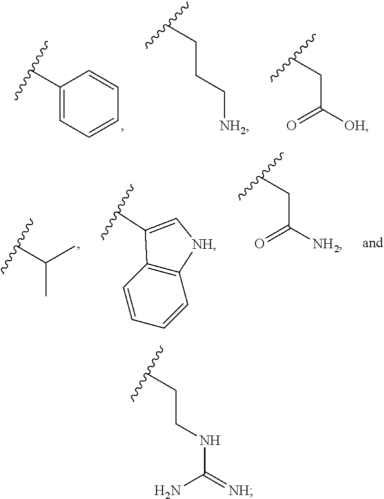

wherein R′ is selected from the group consisting of

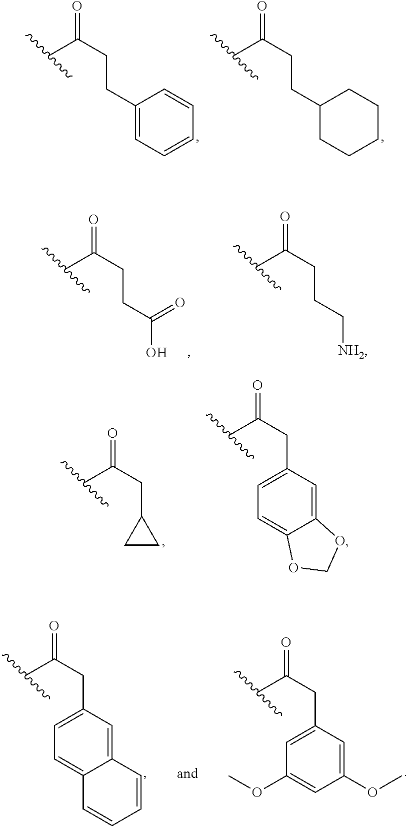

In some embodiments, the γ-AA peptide is formed from four of the γ-AA peptide building blocks. The γ-AA peptide may be selected from the group consisting of Formula (I), Formula (II), Formula (III), Formula (IV), Formula (V), Formula (VI), Formula (VII) and Formula (VIII). In some embodiments, the γ-AA peptide is the compound of Formula (VI).

In a further embodiment, a method of inducing E6 associated protein (E6AP) substrate protein degradation in a cell is presented comprising contacting the cell with a therapeutically effective amount of at least one cyclic γ-substituted-N-acylated-N-aminoethyl amino acids (γ-AA peptide) compound wherein the γ-AA peptide induces E6AP substrate protein degradation by proteosome in the cell. The γ-AA peptide also enhances ubiquitination of E6 associated protein (E6AP) substrate proteins in the cell.

In some embodiments, the γ-AA peptide is formed from four γ-AA peptide building blocks. Each of the four γ-AA building blocks may have the structure of Formula (IX) comprising:

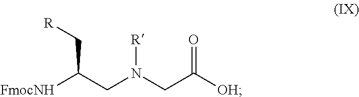

wherein R is selected from the group consisting of H,

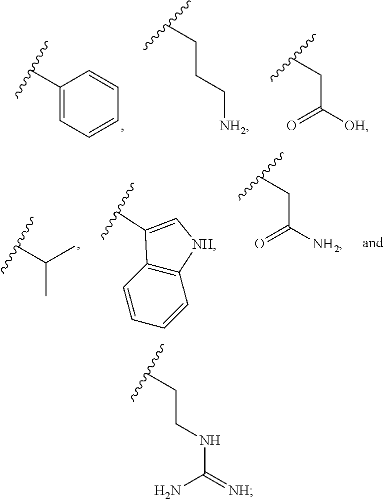

wherein R′ is selected from the group consisting of

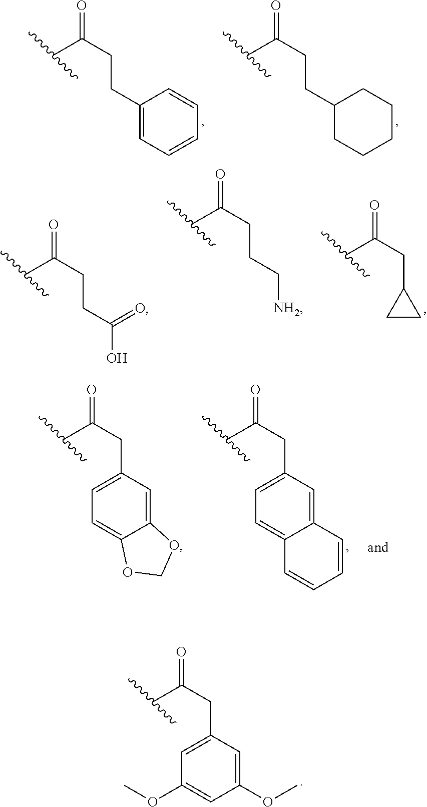

In some embodiments, the γ-AA peptide is selected from the group consisting of Formula (I), Formula (II), Formula (III), Formula (IV), Formula (V), Formula (VI), Formula (VII) and Formula (VIII). In some embodiments, the γ-AA peptide is the compound of Formula (VI).

### BRIEF DESCRIPTION OF THE DRAWINGS

For a fuller understanding of the invention, reference should be made to the following detailed description, taken in connection with the accompanying drawings, in which:

FIG. 1 is an image depicting E6AP-catalyzing polyubiquitination of target proteins in E6-independent and -dependent manners.

FIG. 2A-B are a series of images depicting screening of γ-AA peptide ligands with binding affinities with the E6AP HECT domain. (a) General structures of L-α-peptide and L-γ-AA peptide. (b) γ-AA building blocks used for library preparation.

FIG. 2C-D are a series of images depicting screening of γ-AA peptide ligands with binding affinities with the E6AP HECT domain. (c) Beads bounded with AlexaFluor488-conjugated anti-Flag antibody emitted with green fluorescence. (d) The structure of P6 ligand from the affinity screen.

FIG. 2E is an image depicting screening of γ-AA peptide ligands with binding affinities with the E6AP HECT domain. (e) Fluorescence polarization assay for measuring the binding affinity of P6 with the HECT domain of E6AP. The P6 peptide was labeled with the FITC fluorophore and the K_{d} of the P6-HECT complex was determined to be 218.0±62.3 nM, Error bars=standard deviation from three independent experiments.

FIG. 3 is an image depicting the mechanism of UB transfer activated by P6 peptide leading to accelerated degradation of E6AP substrates.

FIG. 4A-N are a series of images depicting the procedure to synthesize the OBOC T-AA peptide library. (a) Soak in water overnight, then washed with 1:1 (v/v) DCM/Et₂O. (b) (Boc)₂O (0.5 equiv.) in 1:1 (v/v) DCM/Et₂O for 3 h, then washed with DCM and DMF. (c) Fmoc-Met-OH (0.5 equiv.), HOBt (2 equiv.) and DIC (2 equiv.) in DMF for 4 h. (d) 20% piperdine in DMF, then split the beads into five portions equally. (e) Dde protected amino acids (2 equiv.) with PyBOP (6 equiv.) and NEM (6 equiv.) in DMF were added to five peptide synthesis vessels, respectively. (f) TFA/triisopropylsilane/H₂O/Thioanisole (v:v:v:v 94:2:2:2). (g) Fmoc protected γ-AA peptide/HOBt/DIC (3:6:6 equiv.) in DMF were added to five peptide synthesis vessels, respectively. for 4 h. (h) Pd(PPh₃)₄ (1%) and Me₂NH·BH₃ (10%) in DCM for 10 min. (i) Dmt protected mercaptopropionic acid/HOBt/DIC (3:6:6 equiv) in DMF overnight. (j) NH₂OH·HCl and of imidazole in NMP, diluted with DCM. (k) 20% piperdine in DMF. (1) 4-(bromomethyl)benzoyl chloride (2 equiv.) and DIPEA (4 equiv.) in DCM for 1 h. (m) TFA/triisopropylsilane/DCM (2:2:96; v/v/v). (n) (NH₄)₂CO₃ (10 equiv.) in 1:1 (v/v) DMF/H₂O overnight.

FIG. 4O is an image of the decoding map.

FIG. 5A-H are a series of images depicting a schematic illustration of the screening method. (a) The beads were incubated with blocking buffer (1% BSA in Tris buffer with a 1000× excess of E. coli lysate) for 1 h, then washed with Tris buffer and incubated with the AlexaFluor488-conjugated FLAG Epitope tag monoclonal antibody for 2 h at room temperature. After being washed with Tris buffer, then the beads were transferred into a 6-well plate. (b) Beads were observed under a fluorescence microscope. (c) Beads emitting green fluorescence were picked up and discarded, The rest of the beads were pooled into a peptide vessel. (d) The beads were treated with 8 M guandine-HC for 1 h to remove the bond protein. After wash and equilibration in Tris buffer, the beads were incubated with Flag-tagged E6AP domain at a concentration of 50 nM for 4 h with 1% BSA in Tris buffer and 1000× excess of E. coli lysate. After the thorough wash with Tris buffer, the library beads were incubated with AlexaHuor488-conjugated FLAG Epitope tag monoclonal antibody at room temperature for 2 h. (e) The beads were transferred into a 6-well plate to be observed under a fluorescence microscope. (f) The beads emitting green fluorescence were picked up as putative hits. (g) Cleavage the decoding sequence fron the beads. (h) The cleaved peptide was dissolved in ACN:H₂O (4:1) and decoded by MALDI/MS.

FIG. 6A is a series of images depicting the MALDI/MS of the decoding sequence and the structure of corresponding peptides P1 and P2. H* stands for the homoserine lactone.

FIG. 6B is a series of images depicting the MALDI/MS of the decoding sequence and the structure of corresponding peptides P3 and P4. H* stands for the homoserine lactone.

FIG. 6C is a series of images depicting the MALDI/MS of the decoding sequence and the structure of corresponding peptides P5 and P6. H* stands for the homoserine lactone.

FIG. 6D is a series of images depicting the MALDI/MS of the decoding sequence and the structure of corresponding peptide P7. H* stands for the homoserine lactone.

FIG. 6E is a series of images depicting the MALDI/MS of the decoding sequence and the structure of corresponding peptide P8. H* stands for the homoserine lactone.

FIG. 7 is a table depicting the alignment of the peptide sequences from the library screening and the binding affinities of the identified γ-AA peptide ligands P1-8 with the HECT domain of E6AP as measured by fluorescence polarization assay. Positive-charged residues were shown in blue and negative-charged residues were shown in red. a and b denote the chiral and achiral side chains in a γ-AA building block, respectively.

FIG. 8A-D are a series of images depicting binding affinity experiments with FITC labeled peptides bound to HCT domain of E6AP assessed by fluorescence polarization assay. (a) P1, (b) P2, (c) P3, and (d) P4.

FIG. 8E-H are a series of images depicting binding affinity experiments with FITC labeled peptides bound to HCT domain of E6AP assessed by fluorescence polarization assay. (e) P5, (f) P6, (g) P7, and (h) P8.

FIG. 9A-C are a series of images depicting the effect of γ-AA peptide ligands on the UB ligase activity of E6AP assayed by self-ubiquitination. (a) Assaying the effects of ligands P1-8 on E6AP catalysis based on the self-ubiquitination of the HECT domain of E6AP. HECT domain self-ubiquitination was measured by Western blot probed with an anti-Flag antibody that binds to the N-terminal Flag tag fused to the HECT domain. The P6 ligand showed a stimulatory effect on the HECT domain activity. (b) Assaying the effect of P1-8 on E6AP activity based on the self-ubiquitination of the full-length E3. Ubiquitination of E6AP was followed by probing the N-terminal Flag tag fused to the E3 on the Western blot. (c) Dose-dependent activation of the HECT domain (left panel) and full-length E6AP (right panel) by the P6 ligand as measured by the self-ubiquitination reaction.

FIG. 10A-C are a series of images depicting the effect of the γ-AA peptide ligands on the UB ligase activity of E6AP assayed by substrate ubiquitination. (a) Measuring the effects of ligands P1-8 on UbxD8 ubiquitination catalyzed by E6AP. The ubiquitination reaction contains 10 or 100 μM of the ligands incubating with the Uba1-UbcH7-E6AP cascade and UbxD8 as the E6AP substrate. Ligand P6 showed the most significant stimulatory effect on UbxD8 ubiquitination catalyzed by E6AP. (b) Dose-dependent activation of E6AP-catalyzed substrate ubiquitination by the P6 ligand. Varying concentrations of P6 was added to the ubiquitination reaction containing the Uba1-UbcH7-E6AP cascade and the E6AP substrates UbxD8 (left panel), HHR23A (middle panel) and β-catenin (right panel). Ubiquitination of the substrates was measured by Western blots of the reaction probed with antibodies specific for each substrate. (c) Ligands P1-8 have no stimulatory effect on p53 ubiquitination catalyzed by E6AP pairing with the E6 protein of HPV. 10 or 100 μM of the ligands were incubated with the E6AP enzymatic cascade, the viral E6 protein, and p53. The ubiquitination of p53 was followed by a Western blot of the reaction mixture probed with an anti-p53 antibody.

FIG. 11A-B are a series of images depicting the stimulatory effect of the P6 ligand on E6AP-catalyzed substrate ubiquitin in the cell and acceleration of the degradation of E6AP substrates. (a) P6 enhanced the ubiquitination of E6AP substrates UbxD8 (left panel) and HHR23A (right panel) in HEK293 cells. P6 peptide of 0, 25 and 50 μM was incubated with HEK293 cells for 14 h and the cells were treated with proteasome inhibitor MG132 for another 4 h. The cells were harvested for immunoprecipitating the substrate proteins with specific antibodies from the cell lysate, and the ubiquitination levels of the substrate proteins were measured by Western blots probed with an anti-UB antibody. *, IgG light chain (LC); **, IgG heavy chain (HC). (b) Cycloheximide (CHX) chase assay to measure the accelerated degradation of UbxD8 (left panels) and HHR23A (middle panels) in the presence of varying concentrations of P6. β-actin levels in the cell were measured in parallel as a control (right panels). Cells were incubated with varying concentrations of P6 peptide for 14 h and treated with ribosome inhibitor CHX for 0, 1, and 3 hours before harvesting to collect cell lysates. Levels of UbxD8, Rad23A and 0-actin in the cell lysates were measured by Western blot probed with specific antibodies.

FIG. 11C is a series of graphs depicting the stimulatory effect of the P6 ligand on E6AP-catalyzed substrate ubiquitin in the cell and acceleration of the degradation of E6AP substrates. (c) Levels of the E6AP substrates UbxD8 (top panel) and HHR23A (bottom panel) were plotted against the chase time after the cells were incubated with varying concentrations of the P6 peptide. P6 accelerated UbxD8 degradation at a concentration of 5 μM and accelerated Rad23A degradation at a concentration of 1 μM, Error bars=standard deviation from three independent experiments.

FIG. 12A is a series of images depicting analytic HPLC traces of P6 incubation with ammonium bicarbonate buffer or incubation with pronase (0.1 mg/mL) for 24 hours. Specifically, the top picture is the trace of pronase with a partial zoom from 8.5 min to 23.5 min, the middle picture is the trace of P6 after incubation with ammonium bicarbonate buffer and a partial zoom from 10 min to 34 min was included, and the bottom picture is the trace of P6 after incubation with pronase and a partial zoom from 6 min to 36 min was included.

FIG. 12B is a series of images depicting the serum stability of P6 in 25% serum (v/v) at 37° C. for 24 hours. Specifically, the top picture is the trace of P6 after incubation with water for 24 h. the bottom picture is the trace of P6 after incubation with 25% serum for 24 h.

FIG. 13A-B are a series of images depicting HPLC trace of peptide P1 and P2.

FIG. 13C-D are a series of images depicting HPLC trace of peptide P3 and P4.

FIG. 13E-F are a series of images depicting HPLC trace of peptide P5 and P6.

FIG. 13G-H are a series of images depicting HPLC trace of peptide P7 and P8.

FIG. 14A-B are a series of images depicting HPLC trace of FITC-labeled peptide P1 and P2.

FIG. 14C-D are a series of images depicting HPLC trace of FITC-labeled peptide P3 and P4.

FIG. 14E-F are a series of images depicting HPLC trace of FITC-labeled peptide P5 and P6.

FIG. 14G-H are a series of images depicting HPLC trace of FITC-labeled peptide P7 and P8.

FIG. 15A-B are chemical structures of FITC-labeled peptides P1 and P2.

FIG. 15C-D are chemical structures of FITC-labeled peptides P3 and P4.

FIG. 15E-F are chemical structures of FITC-labeled peptides P5 and P6.

FIG. 15G-H are chemical structures of FITC-labeled peptides P7 and P8.

FIG. 16 is a table for HRMS of all peptides and FITC-labeled peptides.

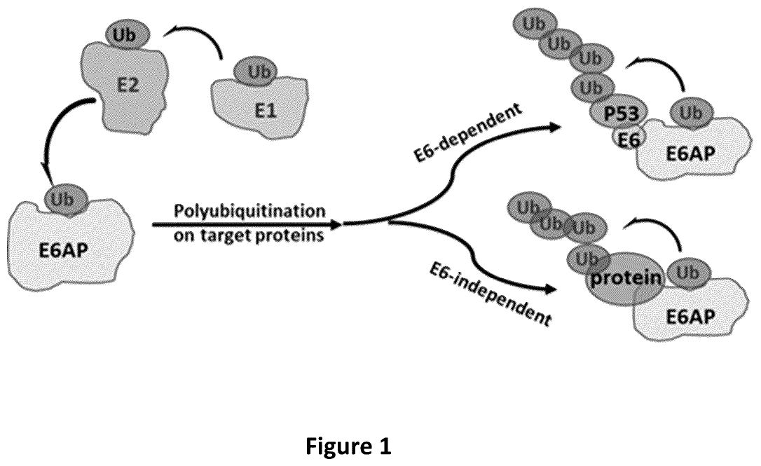

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Abbreviations

PROTAC—proteolysis-targeting chimeras
E6AP—E6 associated protein
HPV—human papillomavirus
γ-AA peptide—γ-substituted-N-acylated-N-aminoethyl peptide
PNA—peptide nucleic acids
HECT domain—homologous to the E6AP Carboxyl Terminus
OBTC—one-bead-two-compound
MALDI—matrix-assisted laser desorption/ionization
MS/MS—tandem mass spectrometry
Uba1—Ubiquitin Like Modifier Activating Enzyme 1
UbcH7—Ubiquitin-conjugating enzyme E2
MG132—carbobenzoxy-Leu-Leu-leucinal
CHX chase—cycloheximide chase
GLP-1—glucagon-like peptide 1
MDM2—mouse double minute 2 homolog
BCL9—the B cell lymphoma 9
UB—ubiquitin
HOBt—hydroxybenzotriazole
DMF—dimethylformamide
DIC—N,N′-Diisopropylcarbodiimide;
Alloc—allyloxycarbonyl
Dde—1-(4,4-dimethyl-2,6-dioxocyclohex-1-ylidene)ethyl
PyBop—benzotriazol-1-yloxytripyrrolidinophosphonium hexafluorophosphate
NEM—N-Ethylmaleimide
Tris buffer—tris(hydroxymethyl) aminomethane buffer
BSA—bovine serum albumin
ACN—acetonitrile
FITC—Fluorescein isothiocyanate
DIPEA—N,N-Diisopropylethylamine
DCM—dichloromethane
TFA—trifluoroacetic acid
PMSF—phenylmethylsulfonyl fluoride
DTT—dithiothreitol

### Definitions

As used herein, the singular forms “a,” “an” and “the” include plural referents unless the context clearly dictates otherwise.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

As used herein, “about” means approximately or nearly and in the context of a numerical value or range set forth means±10% of the numerical.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

As used herein “patient” is used to describe an animal, preferably a human, to whom treatment is administered, including prophylactic treatment with the compositions of the present invention. “Patient” is used interchangeably with “subject” herein.

As used herein “animal” means a multicellular, eukaryotic organism classified in the kingdom Animalia or Metazoa. The term includes, but is not limited to, mammals. Non-limiting examples include rodents, mammals, aquatic mammals, domestic animals such as dogs and cats, farm animals such as sheep, pigs, cows and horses, and humans. Wherein the terms “animal” or the plural “animals” are used, it is contemplated that it also applies to any animals.

As used herein, the term “pharmaceutically acceptable carrier” is used to describe any of the standard pharmaceutically acceptable carriers. The pharmaceutically acceptable carrier can include excipients such as diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin EW [1995] Easton Pennsylvania, Mack Publishing Company, 19^{th} ed.) describes formulations which can be used in connection with the subject invention.

Any of the compounds disclosed herein may be administered with or without an excipient. Excipients include, for example, encapsulating materials or additives such as absorption accelerators; antioxidants; binders; buffers; coating agents; coloring agents; diluents; disintegrating agents; emulsifiers; extenders; fillers; flavoring agents; humectants; lubricants; perfumes; preservatives; propellants; releasing agents; sterilizing agents; sweeteners; solubilizers; wetting agents; and mixtures thereof.

As used herein, “administering” or “administration” refers to the process by which the compounds of the present invention are delivered to a subject. The compounds of the present invention may be administered in a variety of ways including, but not limited to, injection into the central nervous system including the brain, including but not limited to, intrastriatal, intrahippocampal, ventral tegmental area (VTA) injection, intracerebral, intracerebroventricular, intracerebellar, intramedullary, intranigral, intraventricular, intracisternal, intracranial, intraparenchymal including spinal cord and brain stem; orally; parenterally (referring to intravenous and intraarterial and other appropriate parenteral routes); intramuscularly; intraperitoneally; intrasternally; intrathecally; subcutaneously; nasally; buccally; and rectally, among others. Any of the compounds may also be delivered through encapsulation in vesicles such as liposomes, niosomes, micelles, etc.

While any disorder characterized by E3 malfunctions is contemplated, the definitions use the example of neurodegenerative or neurodevelopmental diseases/disorders. This in no way is meant to limit the scope of the invention to only neurodegenerative or neurodevelopmental disorders.

“Treatment” or “treating” as used herein refers to any of: the alleviation, amelioration, elimination and/or stabilization of a symptom or characteristic, as well as delay in progression of a symptom of a particular disorder. For example, “treatment” of a disorder characterized by E3 malfunctions, such as a neurodegenerative or neurodevelopmental disease may include any one or more of the following: amelioration and/or elimination of one or more symptoms/characteristics associated with the neurodegenerative disease, reduction of one or more symptoms/characteristics of the neurodegenerative disease, stabilization of symptoms/characteristics of the neurodegenerative disease, and delay in progression of one or more symptoms/characteristics of the neurodegenerative disease.

“Prevention” or “preventing” as used herein refers to any of: halting the effects of the disorder characterized by E3 malfunctions, such as a neurodegenerative disease, reducing the effects of the neurodegenerative disease, reducing the incidence of the neurodegenerative disease, reducing the development of the neurodegenerative disease, delaying the onset of symptoms of the neurodegenerative disease, increasing the time to onset of symptoms of the neurodegenerative disease, and reducing the risk of development of the neurodegenerative disease.

As used herein, the term “therapeutically effective amount” is determined based on such considerations as known in the art including the recipient of the treatment, the recipient's tolerance for the compound, the disorder being treated, the severity of the disorder being treated, the composition containing the compound, the time of administration, the route of administration, the duration of treatment, the potency of the compound, the bioavailability of the compound, the rate of clearance of the compound from the body, and whether or not another active agent is co-administered. The amount of the compound of the instant invention that may be administered to a subject must be effective to achieve a response, including but not limited to, improved survival rate, more rapid recovery, and improvement or elimination of symptoms associated with E3 malfunctions such as in neurodegenerative and neurodevelopmental disorders and diseases. In accordance with the present invention, a suitable single dose size is a dose that is capable of preventing or alleviating (reducing or eliminating) a symptom in a patient when administered one or more times over a suitable time period. One of ordinary skill in the art can readily determine appropriate single dose sizes for systemic administration based on the size of a mammal and the route of administration.

The dosing of compounds and compositions of the present invention to obtain a therapeutic or prophylactic effect is determined by the circumstances of the patient, as known in the art. The dosing of a patient herein may be accomplished through individual or unit doses of the compounds or compositions herein or by a combined or prepackaged or pre-formulated dose of a compounds or compositions. An average 40 g mouse has a brain weighing 0.416 g, and a 160 g mouse has a brain weighing 1.02 g, a 250 g mouse has a brain weighing 1.802 g. An average 400 g rat has a brain weighing 2 g. An average human brain weighs 1508 g, which can be used to direct the amount of therapeutic needed or useful to accomplish the treatment described herein.

The compositions used in the present invention may be administered individually, or in combination with or concurrently with one or more other therapeutics for disorders characterized by E3 malfunctions such as neurodegenerative or neurodevelopmental disorders, specifically UBE3A deficient disorders.

“Neurodegenerative disorder” or “neurodegenerative disease” or “neurological disorder” or “neurodevelopmental disorder” as used herein refers to any abnormal physical or mental behavior or experience where the death or dysfunction of neuronal cells is involved in the etiology of the disorder. Further, in some embodiments, the terms describe neurological disorders or diseases which are associated with UBE3A or E6AP deficiencies, and correspondingly with decreased E6AP activity. Exemplary diseases include Angelman's Syndrome, Huntington's disease, Alzheimer's disease, Parkinson's disease, amyotrophic lateral sclerosis, autistic spectrum disorders, epilepsy, multiple sclerosis, Prader-Willi syndrome, Fragile X syndrome, Rett syndrome and Pick's Disease.

“UBE3A deficiency” as used herein refers to a mutation or deletion in the UBE3A gene, the gene encoding E6AP. Stimulation of E6AP is an avenue in treatment of a neurodegenerative disease associated with a UBE3A deficiency.

“Building block” or “scaffold” as used herein refers to a common chemical structure characterizing a group of molecules. In some embodiments, the building block is a γ-AA peptide scaffold having the structure of Formula (IX).

The term “compound” as used herein refers to a chemical formulation, either organic or inorganic, which induces a desired pharmacological and/or physiological effect on a subject when administered in a therapeutically effective amount. “Compound” is used interchangeably herein with “drug” and “therapeutic agent”. When the compound name disclosed herein conflicts with the structure depicted, the structure shown will supercede the use of the name to define the compound intended.

As used herein, the term “substituent” means positional variables on the atoms of a core molecule that are attached at a designated atom position, replacing one or more hydrogen atoms on the designated atom, provided that the atom of attachment does not exceed the available valence or shared valence, such that the substitution results in a stable compound. Accordingly, combinations of substituents and/or variables are permissible only if such combinations result in stable compounds. Any carbon atom as well as heteroatom with a valence level that appears to be unsatisfied as described or shown herein is assumed to have a sufficient number of hydrogen atom(s) to satisfy the valences described or shown.

For the purposes of this description, where one or more substituent variables for a compound of Formula (IX) encompass functionalities incorporated into a compound of Formula (IX), each functionality appearing at any location within the disclosed compound may be independently selected, and as appropriate, independently and/or optionally substituted.

As used herein, the terms “independently selected,” or “each selected” refer to functional variables in a substituent list that may be attached more than once on the structure of a core molecule, where the pattern of substitution at each occurrence is independent of the pattern at any other occurrence. Further, the use of a generic substituent variable on a core structure for a compound described herein is understood to include the replacement of the generic substituent with species substituents that are included within the particular genus, e.g., aryl may be replaced with phenyl or naphthalenyl and the like, such that the resulting compound is to be included within the scope of the compounds described herein.

As used herein, the term “optionally substituted” means that the specified substituent variables, groups, radicals or moieties represent the scope of the genus and may be independently chosen as needed to replace one or more hydrogen atoms on the designated atom of attachment of a core molecule.

As used herein, the terms “stable compound” or “stable structure” mean a compound that is sufficiently robust to be isolated to a useful degree of purity from a reaction mixture and formulations thereof into an efficacious therapeutic agent.

As used herein, the term “form” means a compound of Formulas (I-IX) selected from a free acid, free base, salt, ester, hydrate, solvate, chelate, clathrate, polymorph, isotopologue, stereoisomer, racemate, enantiomer, diastereomer, or tautomer thereof.

As used herein, the term “prodrug” means that a functional group on a compound of Formulas (I-IX) is in a form (e.g., acting as an active or inactive drug precursor) that is transformed in vivo to yield an active or more active compound of Formulas (I-IX) or a form thereof. The transformation may occur by various mechanisms (e.g., by metabolic and/or non-metabolic chemical processes), such as, for example, by hydrolysis and/or metabolism in blood, liver and/or other organs and tissues. A discussion of the use of prodrugs is provided by V. J. Stella, et. al., “Biotechnology: Pharmaceutical Aspects, Prodrugs: Challenges and Rewards,” American Association of Pharmaceutical Scientists and Springer Press, 2007.

The compounds of Formulas (I-IX) or a form thereof can form salts, which are intended to be included within the scope of this description. Reference to a compound of Formula (I) or a form thereof herein is understood to include reference to salts thereof, unless otherwise indicated. The term “salt(s)”, as employed herein, denotes acidic salts formed with inorganic and/or organic acids, as well as basic salts formed with inorganic and/or organic bases.

The term “pharmaceutically acceptable salt(s)”, as used herein, means those salts of compounds of Formulas (I-IX) or a form thereof described herein that are safe and effective (i.e., non-toxic, physiologically acceptable) for use in mammals and that possess biological activity, although other salts are also useful. All such acid salts and base salts are intended to be included within the scope of pharmaceutically acceptable salts as described herein. In addition, all such acid and base salts are considered equivalent to the free forms of the corresponding compounds for purposes of this description.

The use of the terms “salt,” “solvate,” “ester,” “prodrug” and the like, is intended to apply equally to the salt, solvate, ester and prodrug of enantiomers, stereoisomers, rotamers, tautomers, positional isomers, racemates, isotopologues or prodrugs of the instant compounds.

Unless otherwise stated, structures depicted herein are also meant to include all isomeric (e.g., enantiomeric, diastereomeric, optical, and geometric (or conformational)) forms of the structure or a form thereof (including salts, solvates, esters, and prodrugs and transformed prodrugs thereof); for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms.

The compounds of Formula (I) or a form thereof described herein may include one or more chiral centers, and as such may exist as racemic mixtures (R/S) or as substantially pure enantiomers and diastereomers. The compounds may also exist as substantially pure (R) or (S) enantiomers (when one chiral center is present). In one embodiment, the compounds of Formula (I) or a form thereof described herein are (S) isomers and may exist as enantiomerically pure compositions substantially comprising only the (S) isomer. In another embodiment, the compounds of Formula (I) or a form thereof described herein are (R) isomers and may exist as enantiomerically pure compositions substantially comprising only the (R) isomer. As one of skill in the art will recognize, when more than one chiral center is present, the compounds of Formula (I) or a form thereof described herein may also exist as a (R,R), (R,S), (S,R) or (S,S) isomer, as defined by IUPAC Nomenclature Recommendations.

As used herein, the term “substantially pure” refers to compounds of Formulas (I-IX) or a form thereof consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

As used herein, the term “racemate” refers to any mixture of isometric forms that are not “enantiomerically pure”, including mixtures such as, without limitation, in a ratio of about 50/50, about 60/40, about 70/30, or about 80/20, about 85/15 or about 90/10.

All stereoisomers (for example, geometric isomers, optical isomers and the like) of the present compounds of Formulas (I-IX) or a form thereof (including salts, solvates, esters and prodrugs and transformed prodrugs thereof), which may exist due to asymmetric carbons on various substituents, including enantiomeric forms (which may exist even in the absence of asymmetric carbons), rotameric forms, atropisomers, diastereomeric and regioisomeric forms, are contemplated within the scope of the description herein. Individual stereoisomers of the compounds of Formulas (I-IX) or a form thereof described herein may, for example, be substantially free of other isomers, or may be present in a racemic mixture, as described supra.

The term “isotopologue” refers to isotopically-enriched compounds of Formulas (I-IX) or a form thereof which are identical to those recited herein, but for the fact that one or more atoms are replaced by an atom having an atomic mass or mass number different from the atomic mass or mass number usually found in nature.

One or more compounds of Formulas (I-IX) or a form thereof described herein may exist in unsolvated as well as solvated forms with pharmaceutically acceptable solvents such as water, ethanol, and the like, and the description herein is intended to embrace both solvated and unsolvated forms

As used herein, the term “solvate” means a physical association of a compound of Formulas (I-IX) or a form thereof described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates.

Polymorphic crystalline and amorphous forms of the compounds of Formulas (I-IX) or a form thereof, and of the salts, solvates, esters and prodrugs of the compounds of Formulas (I-IX) or a form thereof, are further intended to be included in the scope of the compounds of Formulas (I-IX) or a form thereof described herein.

Also falling within the scope described herein are the in vivo metabolic products of the compounds of Formulas (I-IX) or a form thereof. Such products may result, for example, from the oxidation, reduction, hydrolysis, amidation, glucuronidation, esterification and the like of the administered compound of Formulas (I-IX) or a form thereof, primarily due to enzymatic processes. Accordingly, the compounds of Formulas (I-IX) or a form thereof described herein include those produced by a process comprising contacting a compound of Formulas (I-IX) or a form thereof described herein with a mammalian tissue or a mammal for a period of time sufficient to yield a metabolic product thereof.

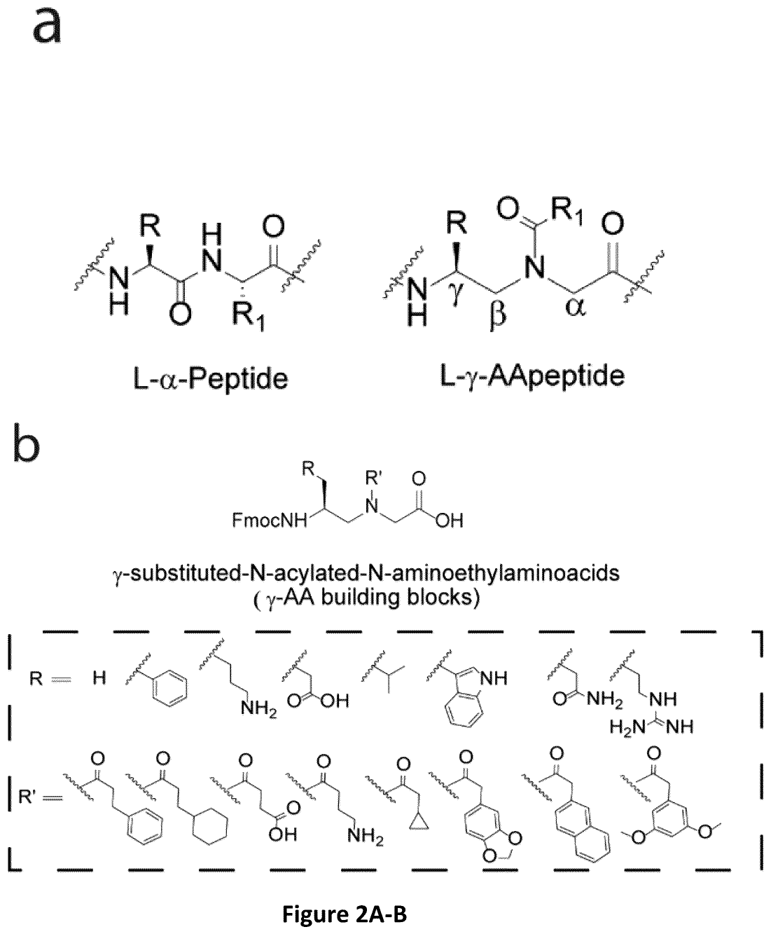

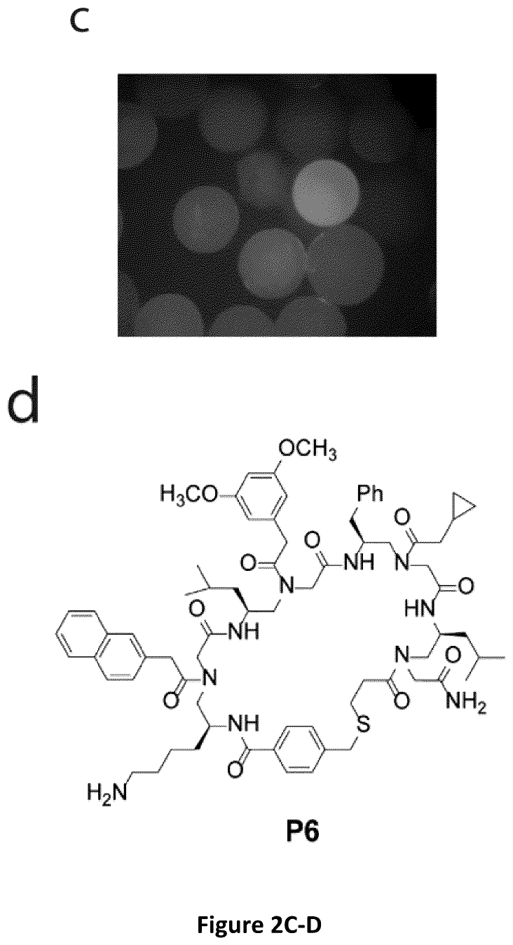

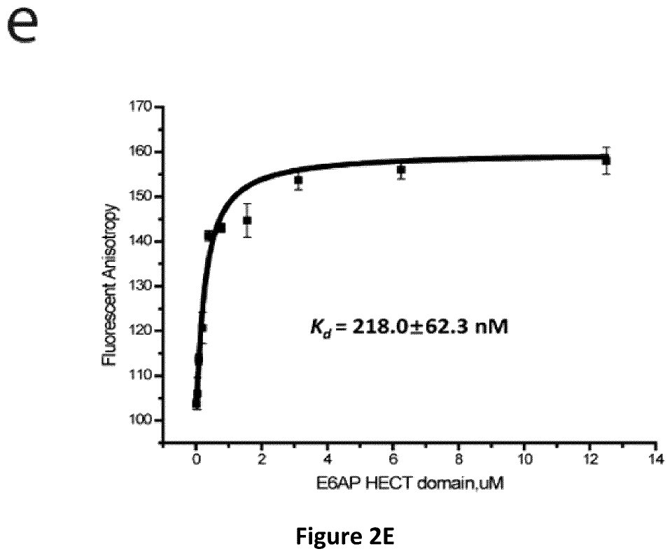

The inventors discovered novel compounds for E6AP activation by screening a γ-AA peptide library for ligands binding to the HECT domain of E6AP that is the catalytic unit of the E3 responsible for transferring UB to the substrate proteins. γ-AA peptides (FIG. 2a), named for the oligomers of γ-substituted-N-acylated-N-aminoethyl amino acids, are derived from the backbone of chiral peptide nucleic acids (PNA). This new class of unnatural peptidomimetics possesses enormous chemical diversity, remarkable resistance to proteolytic degradation, and excellent capacities for cell delivery²⁶.

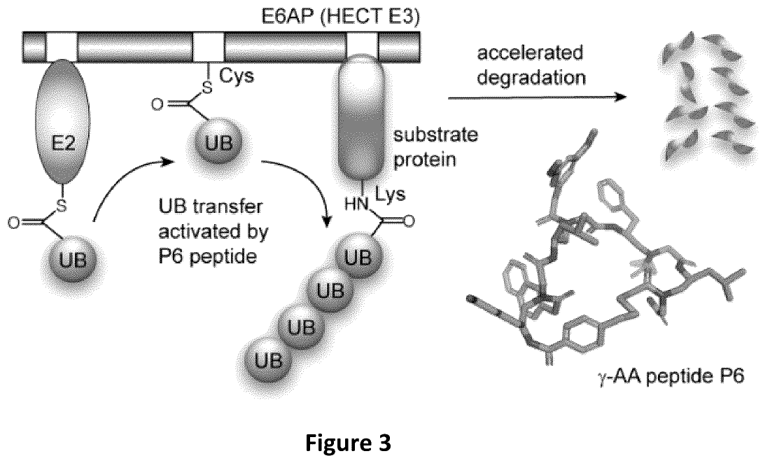

The inventors developed an affinity-based screen for peptides binding to the HECT domain of E6AP. A series of γ-AA cyclic peptides with submicromolar affinity with the target HECT domain was discovered. In particular, one peptide ligand, known as P6, can significantly stimulate the activity of E6AP in ubiquitinating substrate proteins UbxD8, Rad23, and β-catenin in reconstituted reactions. Interestingly, P6 can enhance the ubiquitination of E6AP substrates in the cell and accelerate their degradation by the proteasome. (FIG. 3). The discovery of the γ-AA peptide ligand for E6AP activation attests to the malleability of the non-conventional peptide scaffold for ligand discovery to target the protein ubiquitination cascade. Moreover, the results offer a new therapeutic landscape based on the identification of E3-activating ligands and confirm the feasibility of using E3 activators alongside inhibitors and substrate recruiters for manipulating protein degradation pathways in the cell.

The following non-limiting examples illustrate exemplary compositions and methods of treatment thereof in accordance with various embodiments of the disclosure. The examples are merely illustrative and are not intended to limit the disclosure in any way.

### Example 1—Cyclic γ-AA Peptides can Stimulate E6AP Activity

Library Screening and Characterizing the Affinities of the γ-AA Peptide Ligands with the E6AP HECT Domain

E6AP runs the third relay of the E1-E2-E3 cascade that passes UB to the substrate proteins to form an isopeptide bond between the C-terminal carboxylate of UB and the Lys residues of the substrates. The HECT domain of E6AP engages the UB-E2 thioester conjugate to facilitate the delivery of UB from E2 to a catalytic Cys residue of the HECT domain before passing UB to the substrate proteins²⁷⁻²⁸. The inventors recently engineered the HECT domain of E6AP for the assembly of an orthogonal UB transfer cascade (OUT) to profile the substrate specificity of the E3⁷.

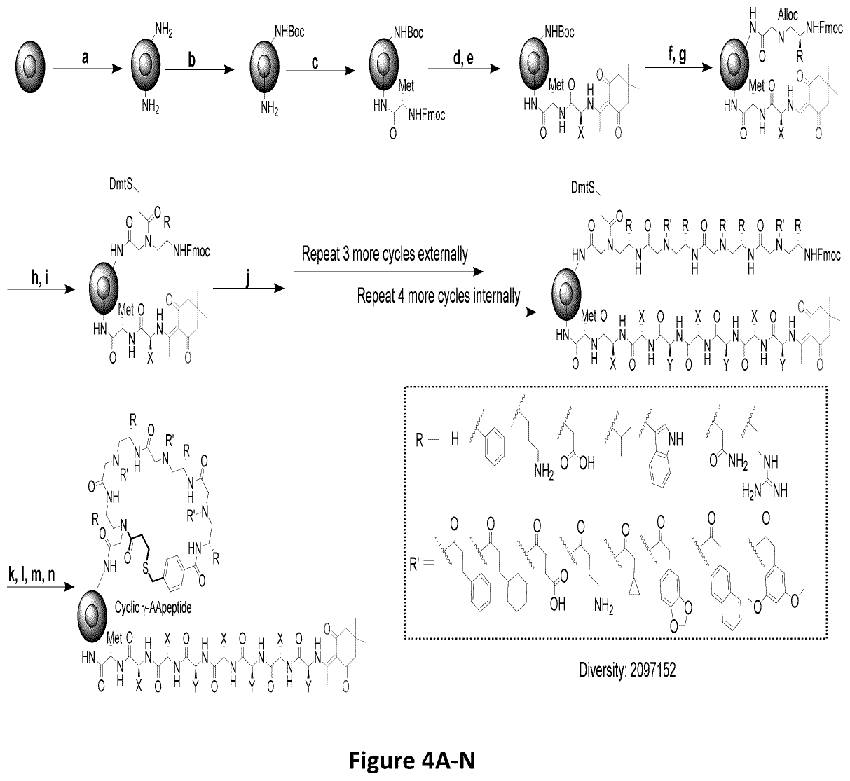

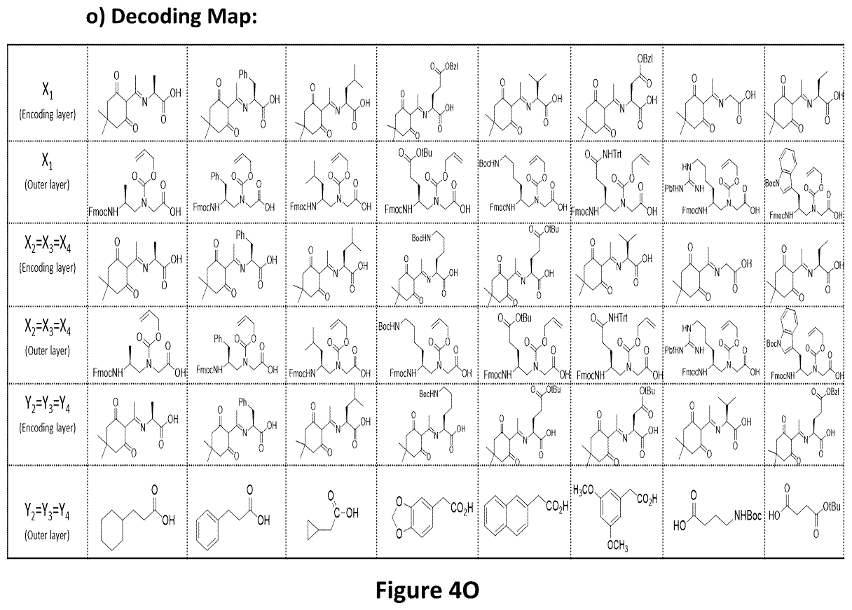

Synthetic ligand binding to the HECT domain of E6AP affects its catalytic activities so they could be further developed as inhibitors or activators of the E3. The inventors designed a cyclic γ-AA peptide library comprising of a variety of γ-AA building blocks (FIG. 2a). Each cyclic γ-AA peptide contained four γ-AA building blocks, and it matched the size of an 8-residue cyclic peptide. The choice of the macrocyclic ring size was based on the inventors previous work that demonstrated high binding affinities of cyclic γ-AA peptides of four units with diverse biological targets²⁹⁻³¹. In the current library design, both side chains of γ-AA building blocks (R and R′) were selected from a pool of hydrophobic and charged groups, and a random combination of the building blocks would constitute a library of greater than 2×10⁶ in diversity (FIG. 2b). A detailed protocol for the preparation of the one-bead-two-compound (OBTC) library of the γ-AA peptides is shown in FIG. 4.

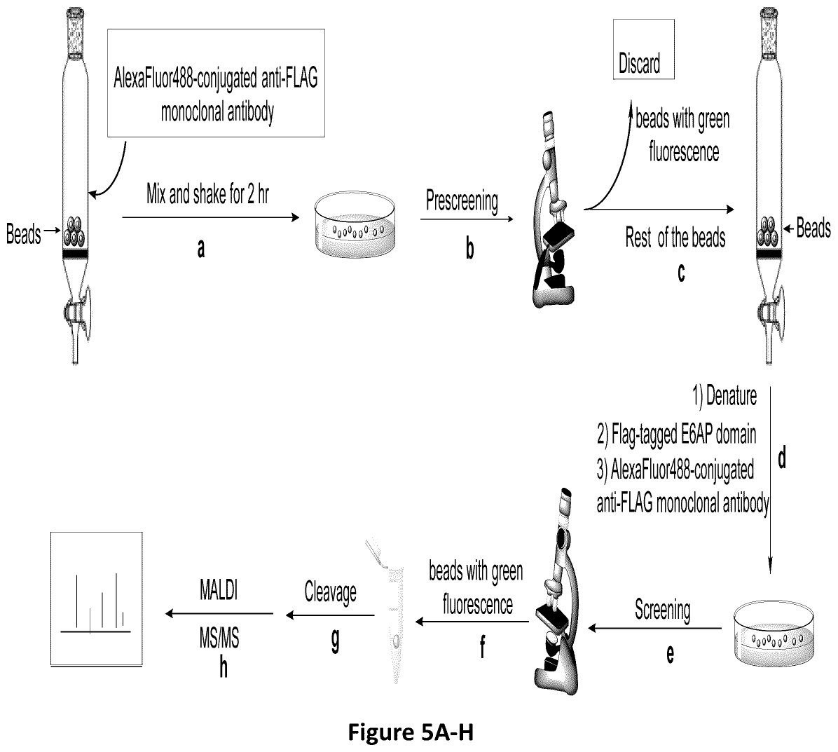

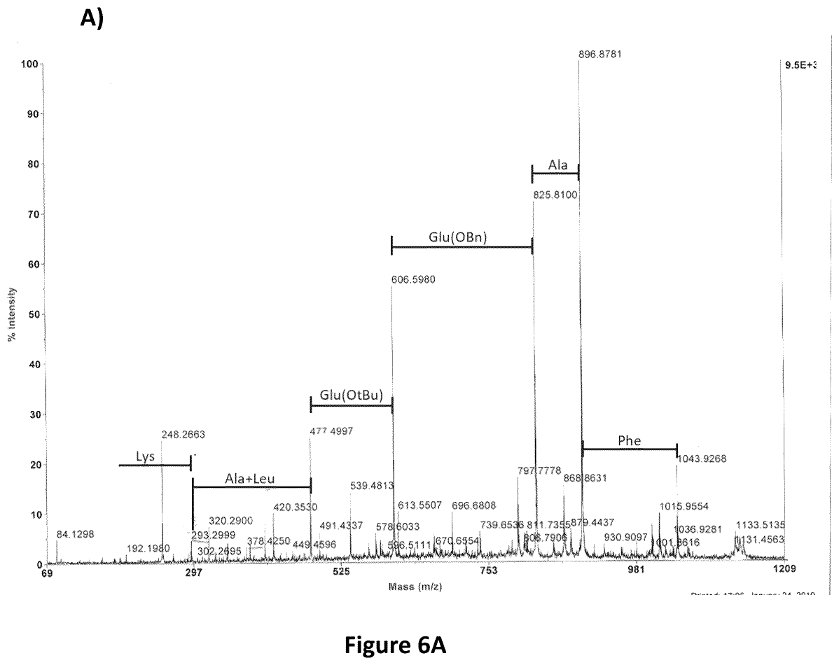

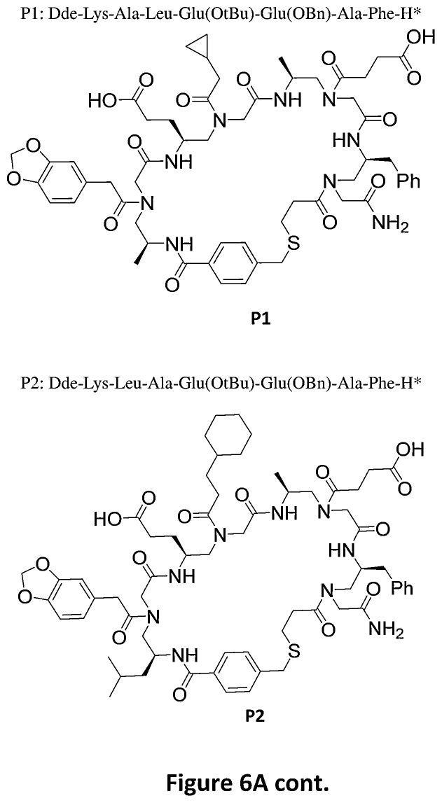

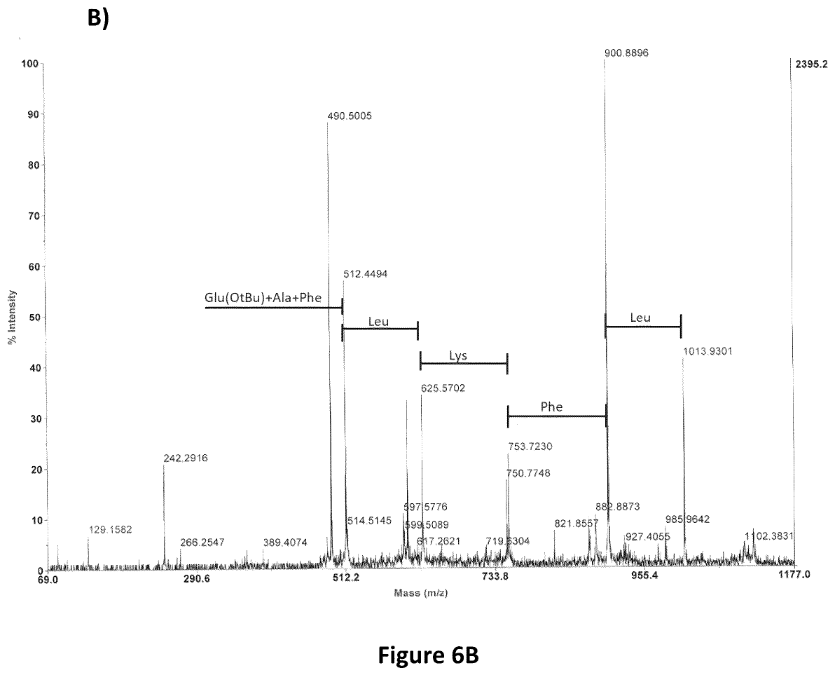

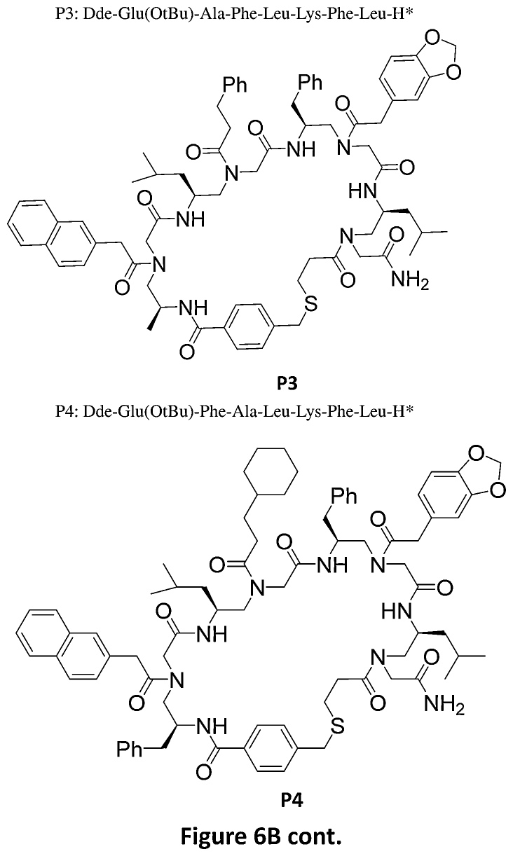

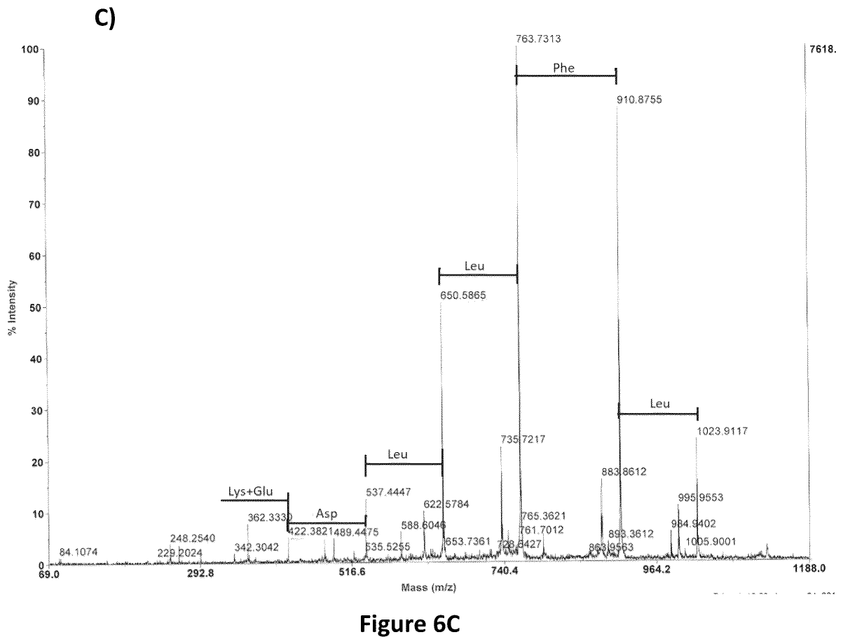

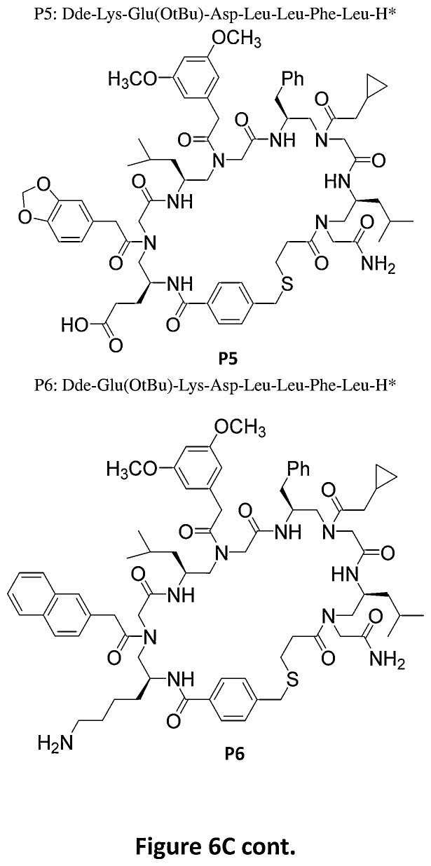

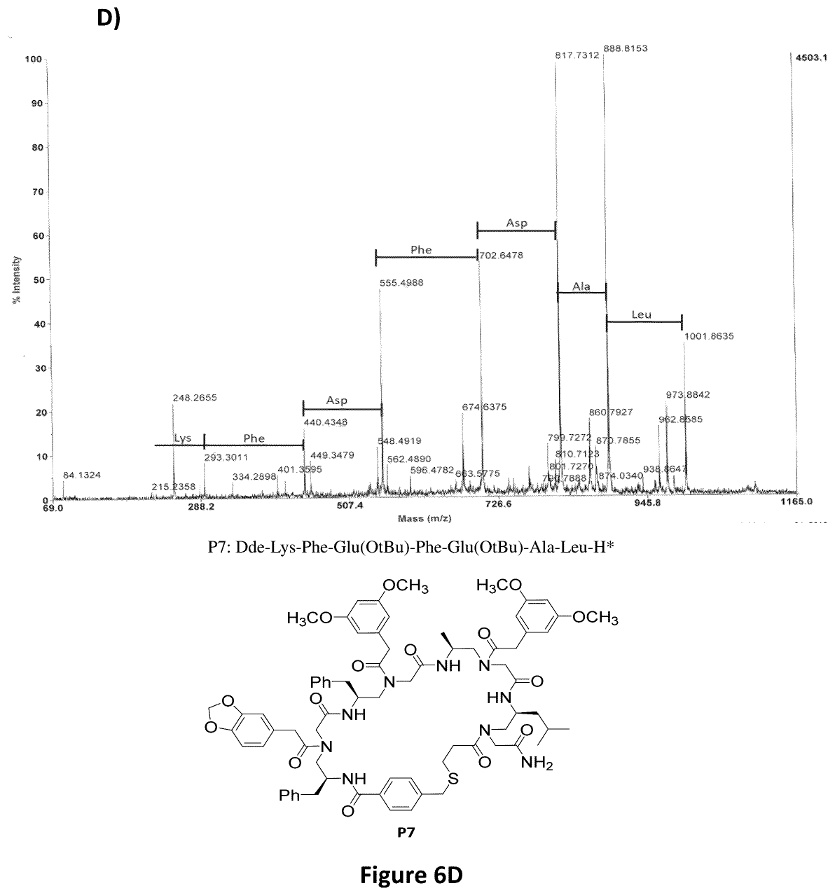

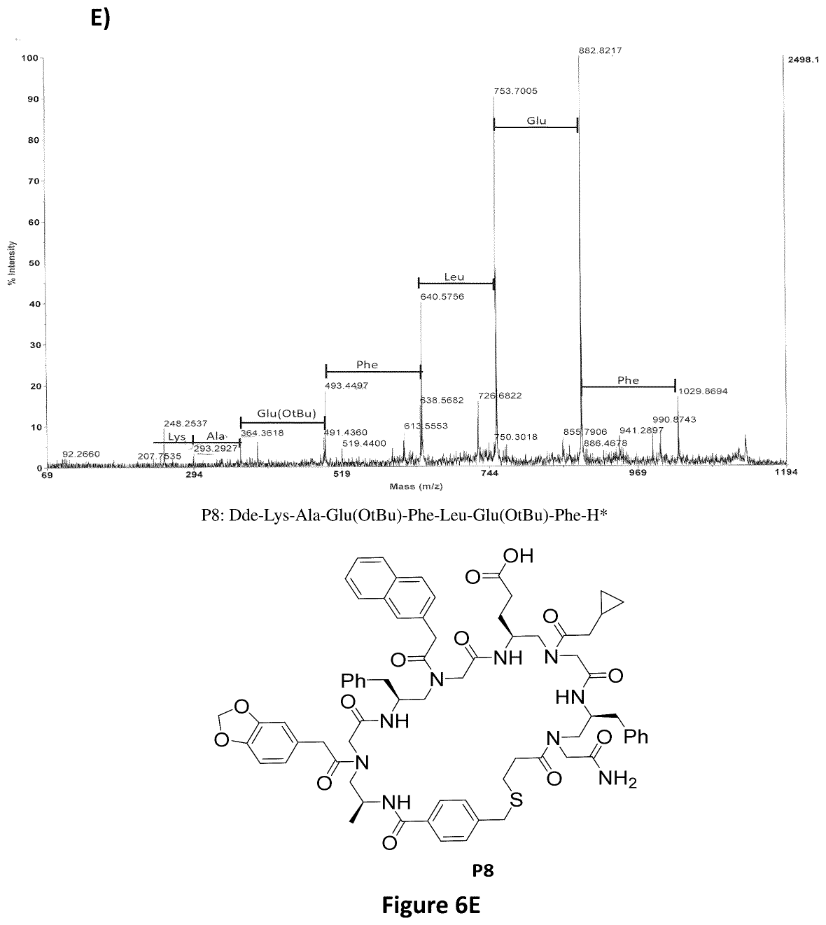

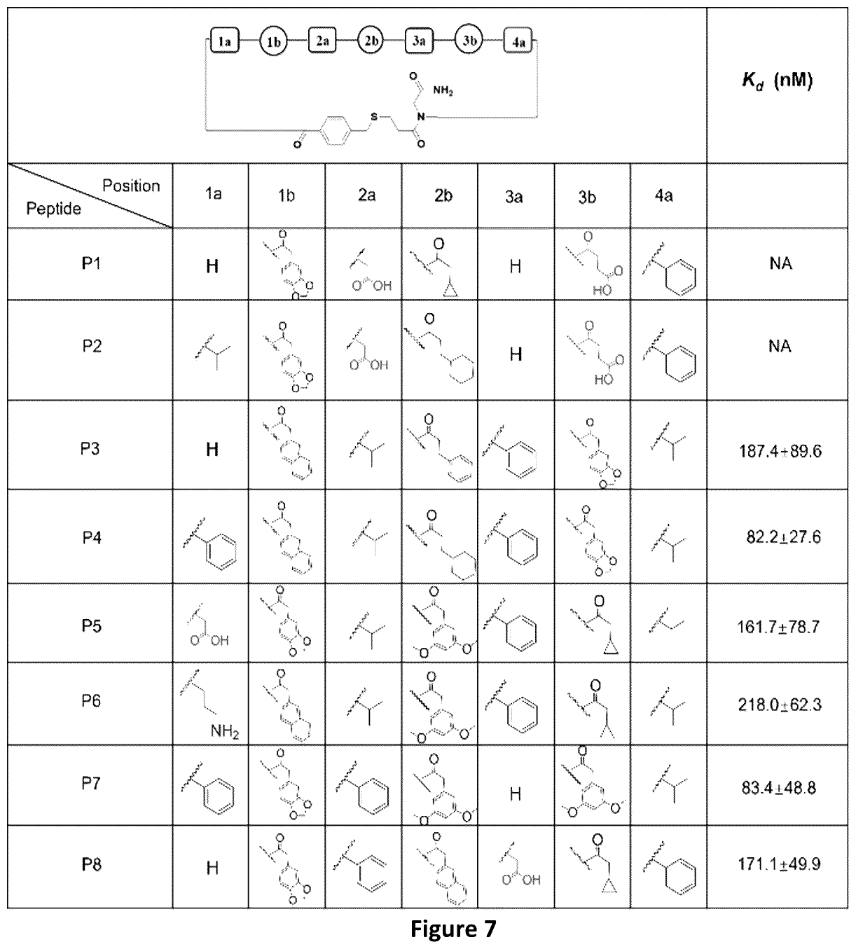

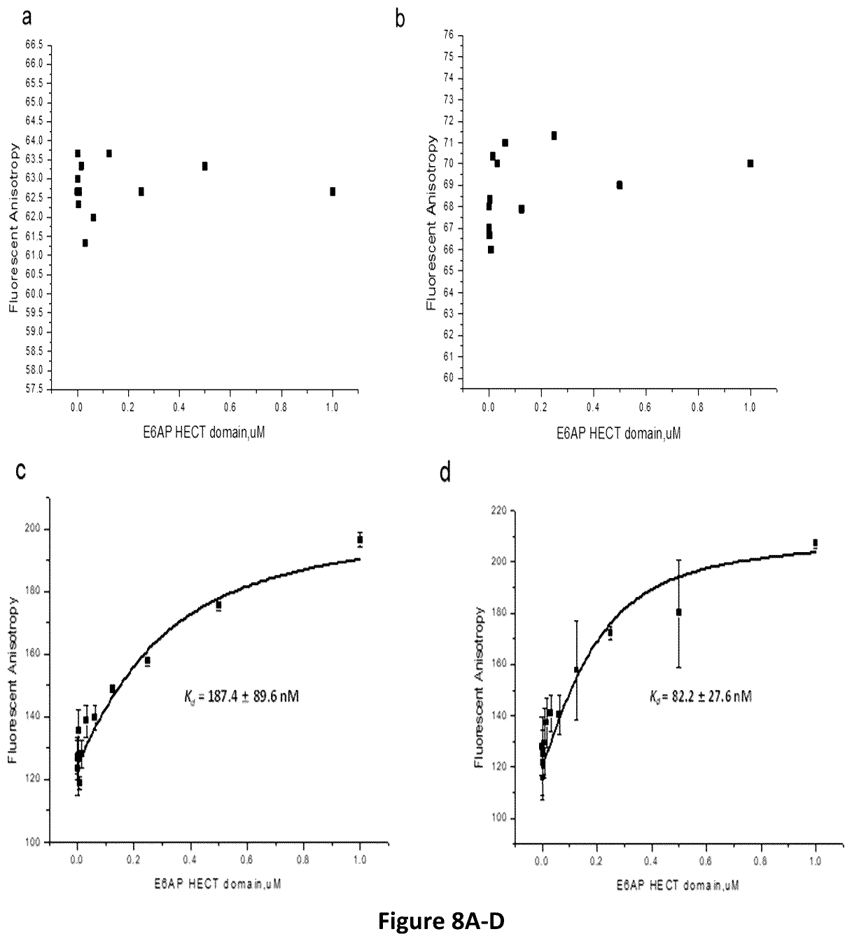

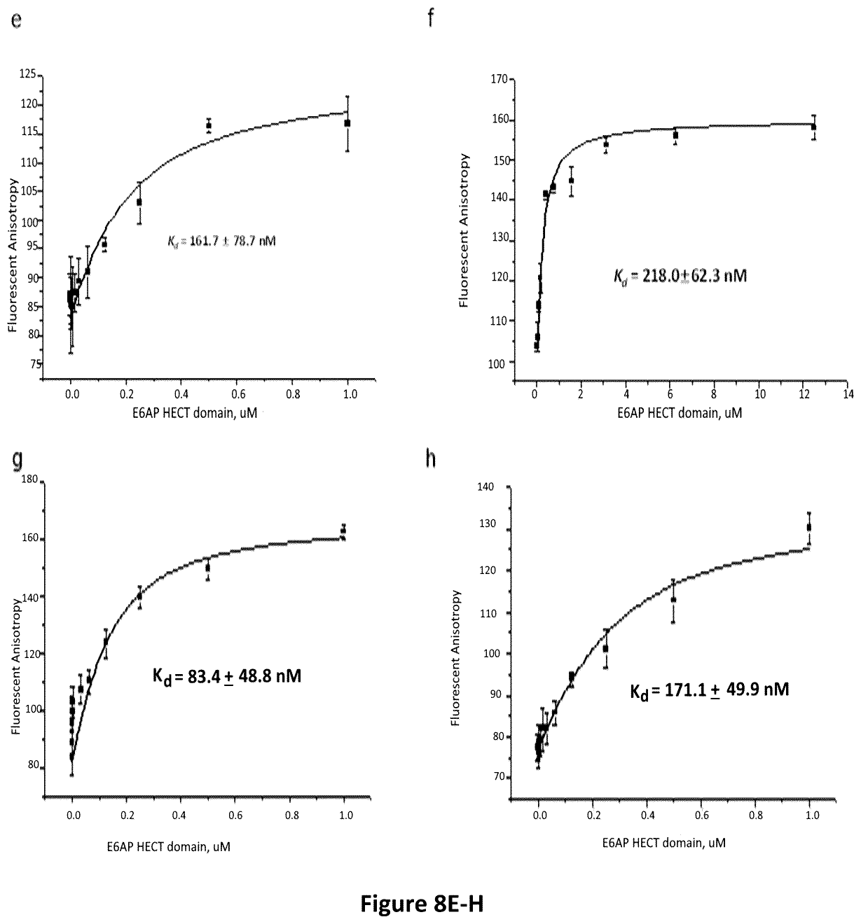

The library was screened based on the binding between the bead-anchored peptides with the HECT domain of E6AP with an N-terminal Flag tag that was recognized by an anti-Flag antibody labeled with AlexaFluor 488 that would emit green fluorescence. The screening protocol is shown in FIG. 5. Five beads with strong green fluorescence were picked for their positive response to the binding of the E6AP HECT domain (FIG. 2c), and the corresponding structures of the γ-AA peptides anchored on the beads were elucidated by the tandem MS/MS of MALDI (FIG. 2d and FIG. 6). The inventors found two beads each yielded a single unambiguous structure, whereas the remaining three beads each was associated with two possible structures, leading to a total of eight γ-AA peptides as putative binders of the E6AP HECT domain. The peptide ligands from the screen were named P1-8 and they were resynthesized to measure their individual binding affinities with the HECT domain by fluorescence polarization (FP). P3-8 were found to have submicromolar binding affinities with the E6AP HECT with K_{d}'s ranging from 80 to 218 nM, while P1 and P2 did not show any measurable binding with the HECT domain (FIG. 7, FIG. 2e and FIG. 8). Alignment of the selected peptide sequences revealed a preference for bulky hydrophobic residues such as naphthyl and 3,4-(methylenedioxy) phenyl at positions 1b, 2b and 3b, and small hydrophobic residues such as isopropyl and phenyl at positions 2a and 4a (FIG. 7). P4 showed a two-fold higher affinity than P3 with the HECT domain while the main difference between the two peptides is that P4 has a phenyl residue at position 1a while P3 has a hydrogen atom at the corresponding position. Similarly, P7 with a phenyl residue at position 1a has a two-fold higher affinity than P8 of a similar structure but with a hydrogen atom at the same position.

### Effects of γ-AA Peptides on E6AP Activity Assayed by Self-Ubiquitination

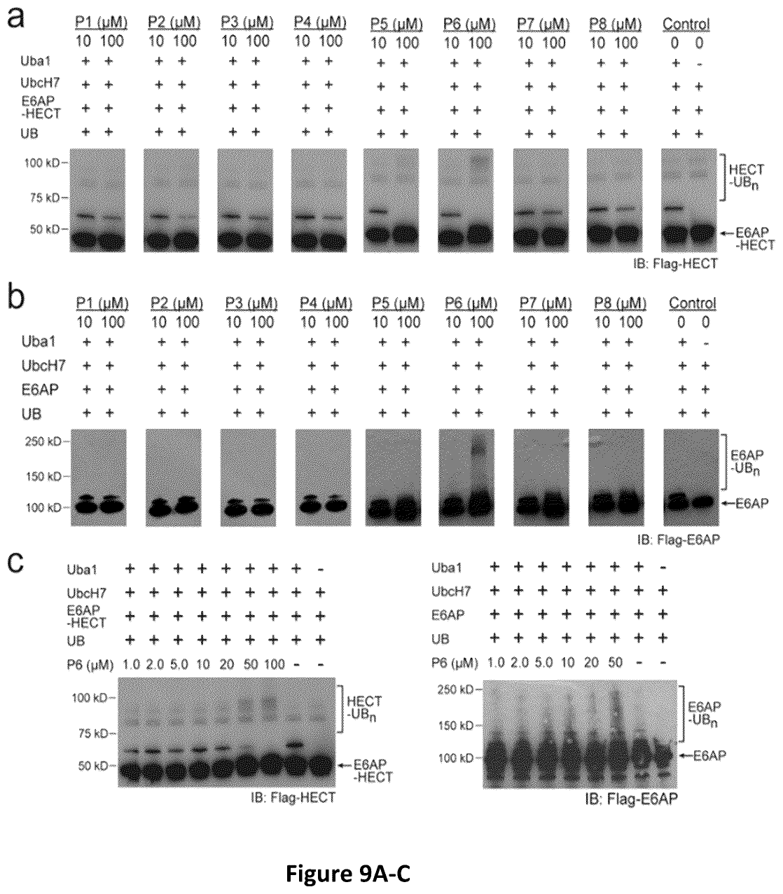

The inventors first assayed the activities of the γ-AA peptides based on their effects on the self-ubiquitination of the HECT domain and full-length E6AP. E6AP HECT was incubated with 10 μM and 100 μM of each peptide from the affinity screen and Uba1 (E1), UbcH7 (E2) and HA-tagged UB (HA-UB) was added to initiate the ubiquitination reaction. Peptides P1-5 and 7-8 had little effect on the formation of UB-HECT conjugate. However, P6 peptide enhanced the formation of polyubiquitinated HECT species in the high-molecular weight range (FIG. 9a). This suggests stimulatory effect of P6 on the catalytic activity of the HECT domain. The assay was repeated on the full-length E6AP with the chosen condition that mainly generated mono-ubiquitinated species of the E3 without the addition of the peptides. Similar to the assay with the HECT domain, there was not much effect of P1-5 and 7-8 on self-ubiquitination of full-length E6AP except that P3 and P4 showed weak inhibitory effect at 100 μM concentration. P6 again stimulated E6AP self-ubiquitination, demonstrating its unique effect on E6AP activation (FIG. 9b). The inventors then assayed the self-ubiquitination of HECT and full-length E6AP in the presence of P6 peptide of varying concentrations and found the peptide can stimulate E3 self-ubiquitination at a concentration of 5 μM (FIG. 9c). Since HECT domain and the full-length E6AP showed a similar response to P6, it is likely that P6 activated E6AP by binding and stimulating the catalytic activity of the HECT domain. It is intriguing that P6 exhibited the weakest binding affinity (218 nM) for the E6AP HECT domain among P3-8 (FIG. 2e), but it has a unique stimulatory effect on the UB transfer reaction catalyzed by the HECT domain. Alignment of the sequences of P6 and other peptides showed P6 has a distinctive positively charged Lys side chain at position 1a while there is a hydrogen (P3 and P8), phenyl (P4 and P7) or Asp sidechain (P5) at the same position (FIG. 7). The Lys side chain at 1a may contribute to the different binding mode of P6 with the HECT domain to activate the catalytic activity of E6AP.

### Effect of γ-AA Peptides on Substrate Ubiquitination Catalyzed by E6AP

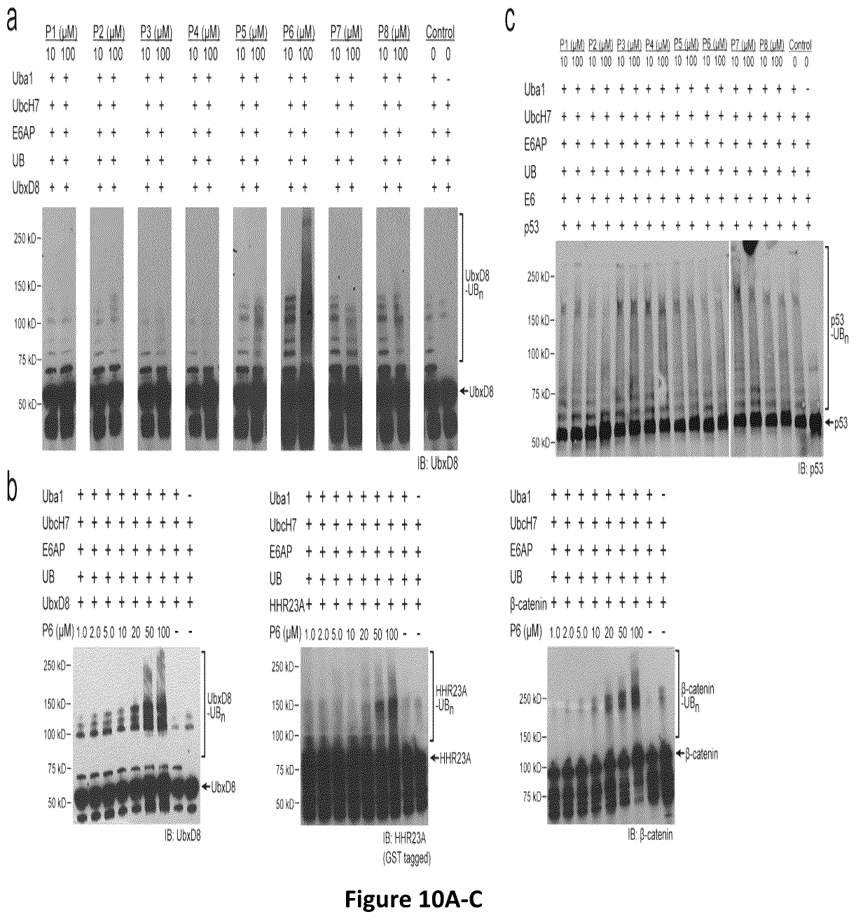

P6 stimulation of E6AP self-ubiquitination prompted the inventors to assay the effect of P6 on the ubiquitination of E6AP substrates. The inventors previously used an engineered-OUT cascade of E6AP to identify UbxD8, an adaptor protein regulating lipid droplet formation, and β-catenin, a transcription factor, as E6AP substrates⁷. Other reports also verified β-catenin and HHR23A, a protein involved in DNA repair, as E6AP substrates³²⁻³⁴. The effect of the peptide ligands on E6AP-catalyzed ubiquitination of UbxD8, HHR23A, and β-catenin was measured. First, E6AP ubiquitination of UbxD8 in the presence of 10 μM and 100 μM peptides was assayed. P1-4 did not show much effect on UbxD8 ubiquitination compared to the control reactions with no addition of the peptides. In contrast, P6 showed a distinctive stimulatory effect on UbxD8 ubiquitination at both concentrations of the peptide, while P5, P7, and P8 showed a weaker stimulatory effect (FIG. 10a). When the ubiquitination assay was performed with varying concentrations of the P6 peptide, all three substrates, including UbxD8, HHR23A, and 0-catenin, showed enhanced ubiquitination in a dose-dependent manner in response to the amount of P6 in the reconstituted reaction (FIG. 10b). These results suggest P6 would enhance the UB-transfer activity of E6AP to a broad range of substrates. The E6 protein from HPV virus can both enhance the activity of E6AP in substrate ubiquitination and stir its substrate specificity to new targets such as p53 11, 35.

The inventors then assayed if the γ-AA peptides from the affinity screen would affect p53 ubiquitination catalyzed by E6AP. The ubiquitination reaction was set up with either 10 μM or 100 μM of the peptides and it was found that none of the peptides, including P6, affected p53 ubiquitination with HPV E6 in the reconstituted reaction (FIG. 10c). Such a result suggests HPV E6 may override the stimulatory effect of P6 on E6AP, so no additional enhancement of p53 ubiquitination by P6 is observed when both P6 peptide and HPV E6 protein were added to the reaction.

### P6-Mediated Enhancement of Ubiquitination and Accelerated Degradation of E6AP Substrates in HEK293 Cells

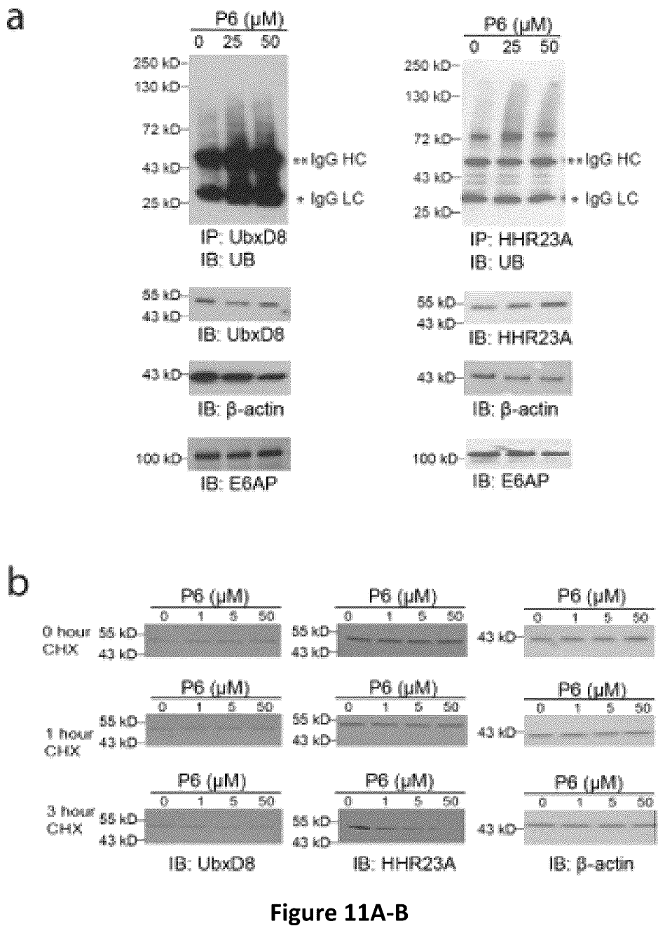

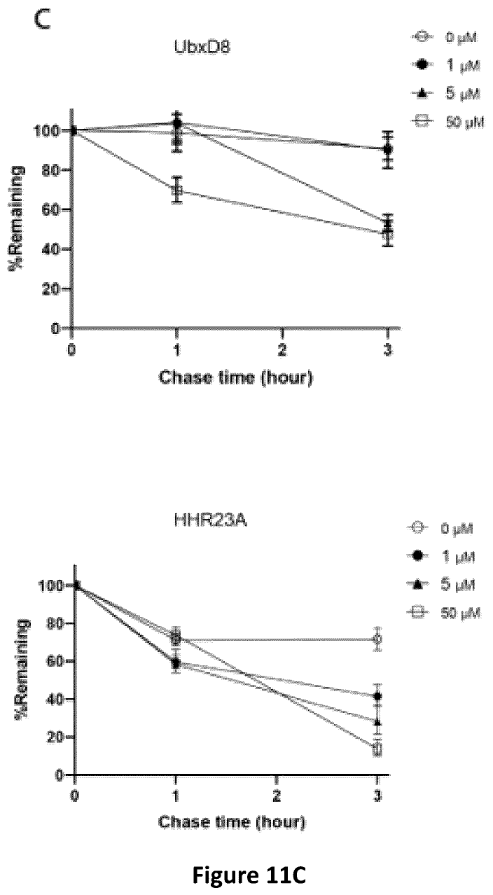

The inventors then assayed if P6 would affect the ubiquitination of E6AP substrates and their stabilities in the cell. HEK293 cells were incubated with 25 μM and 50 μM P6 peptide for 4 hours in the presence of MG132, a proteasome inhibitor to suppress the degradation of ubiquitinated proteins in the cell. The cell was then lysed, UbxD8 and HHR23A immunoprecipitated as E6AP substrates with specific antibodies and the ubiquitination levels of substrate proteins analyzed by Western blotting probed with an anti-UB antibody (FIG. 11a). Both UbxD8 and HHR23A showed an enhanced level of protein ubiquitination in cells with the addition of P6 compared to the control cells with no P6 added. This result suggests that the P6 peptide can enhance the ubiquitination of E6AP substrates in the cell.

To measure if the enhanced E6AP activity due to P6 stimulation would accelerate the degradation of the substrate proteins in the cell, a cycloheximide (CHX) chase assay was done to follow the stability of UbxD8 and HHR23A in the presence of P6. HEK293 cells were pretreated with various concentrations of P6 to stimulate E6AP activity and then CHX, a ribosome inhibitor, was added to block the synthesis of new proteins. At various time points of CHX chase, the cells were lysed and the levels of UbxD8 and HHR23A probed to follow their degradation (Figure l1b). Both substrates showed a faster degradation pattern in cells cultured with 50 μM P6 than in cells with no P6 added. HHR23A also showed accelerated degradation in cells treated with 1 or 5 μM of P6 (FIG. 11c). These results confirm that P6 can promote the degradation of E6AP substrates by stimulating the catalytic activity of E6AP in the cell.

### Stability of P6

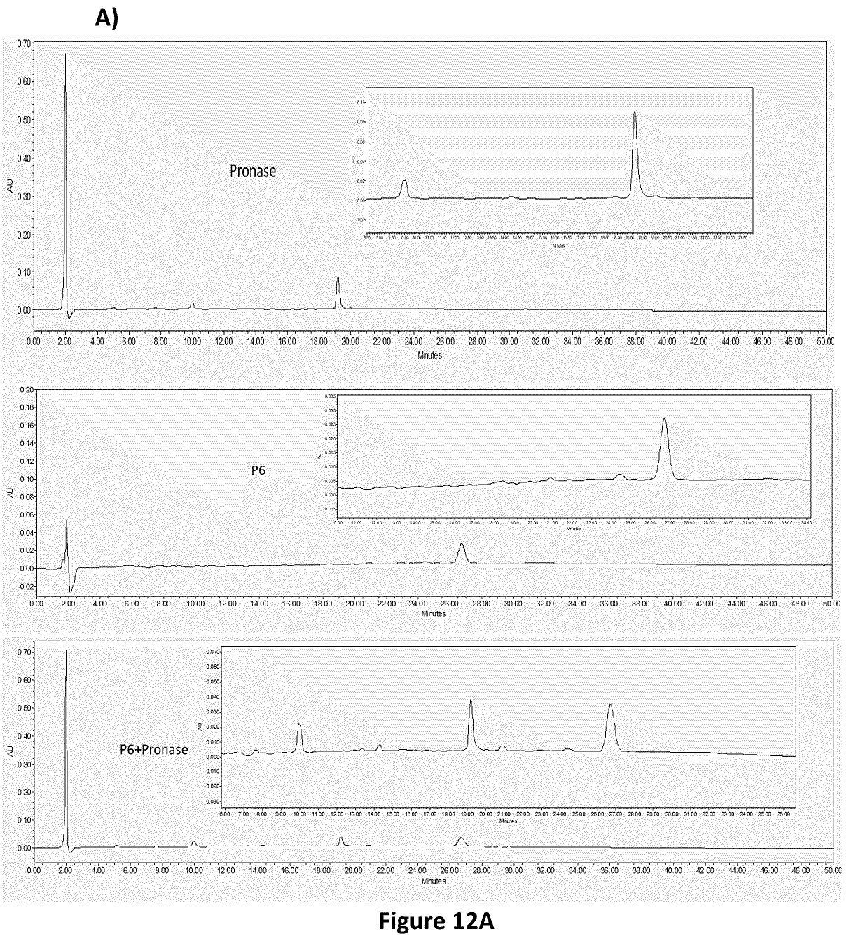

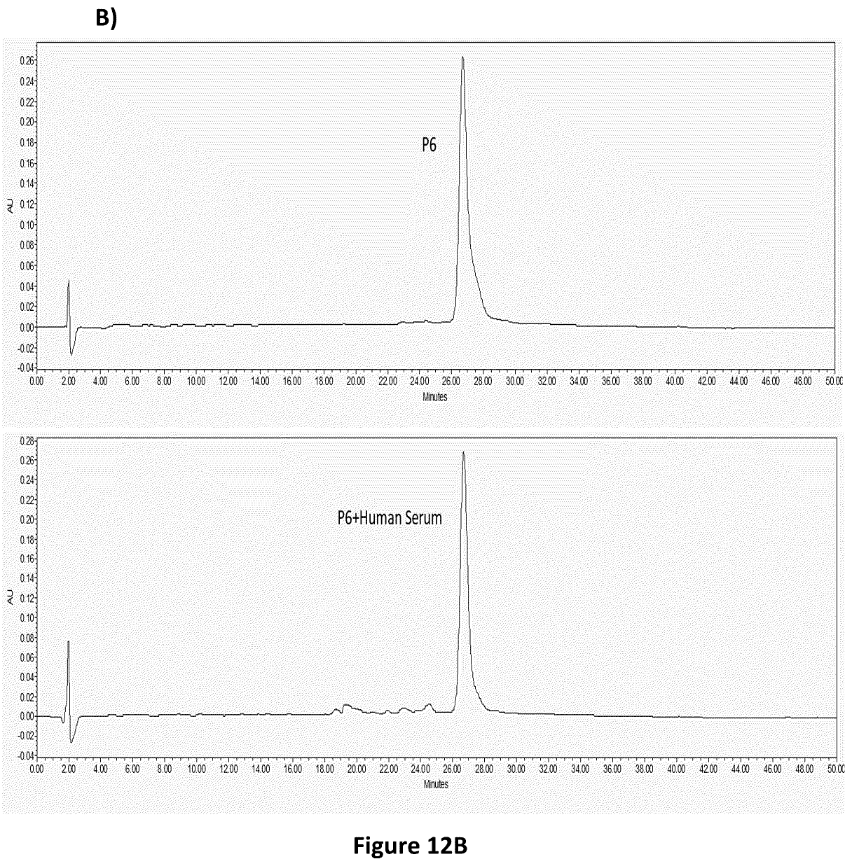

A distinctive characteristic of γ-AA peptides compared with canonical peptides is their remarkable resistance to protease. To assay the stability of P6 in presence of protease, P6 was incubated with 0.1 mg/mL pronase at 37° C. for 24 h. High-performance liquid chromatography and mass spectrometry of the P6 peptide before and after the exposure to pronase showed that P6 was resistant to protease cleavage (FIG. 12a). P6 was also incubated in the human serum for 24 h and its stability was confirmed (FIG. 12b). The superior stability of P6 potentiates its use as a molecular probe for cell-based studies and therapeutic development.

### Materials and Methods

### Reagents

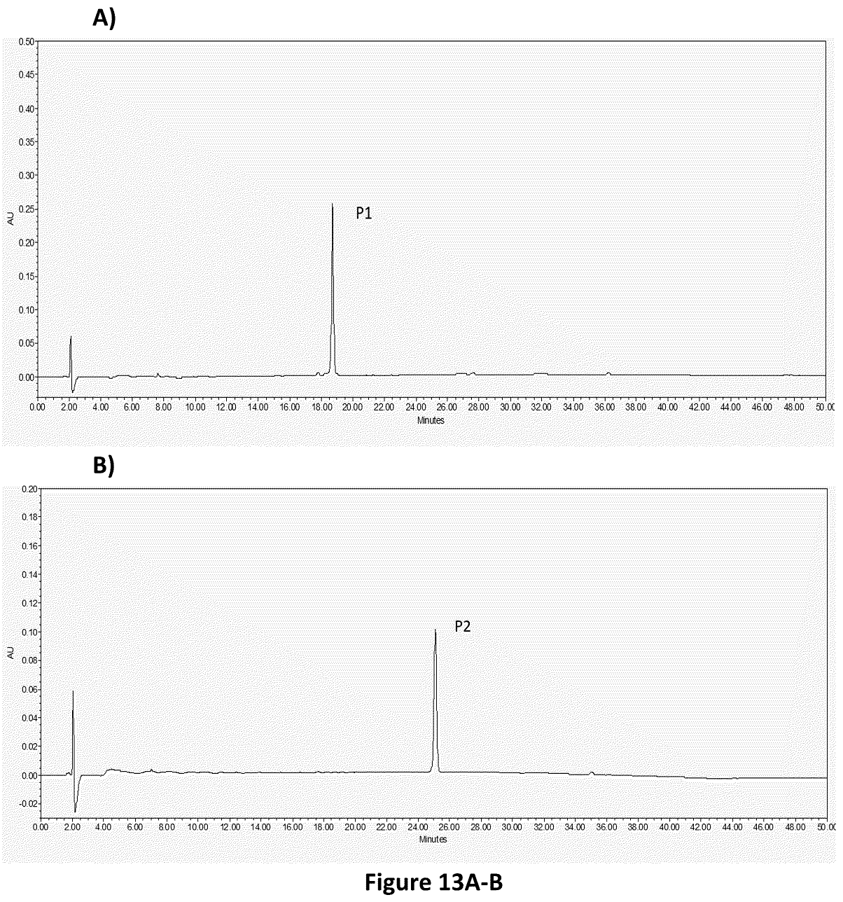

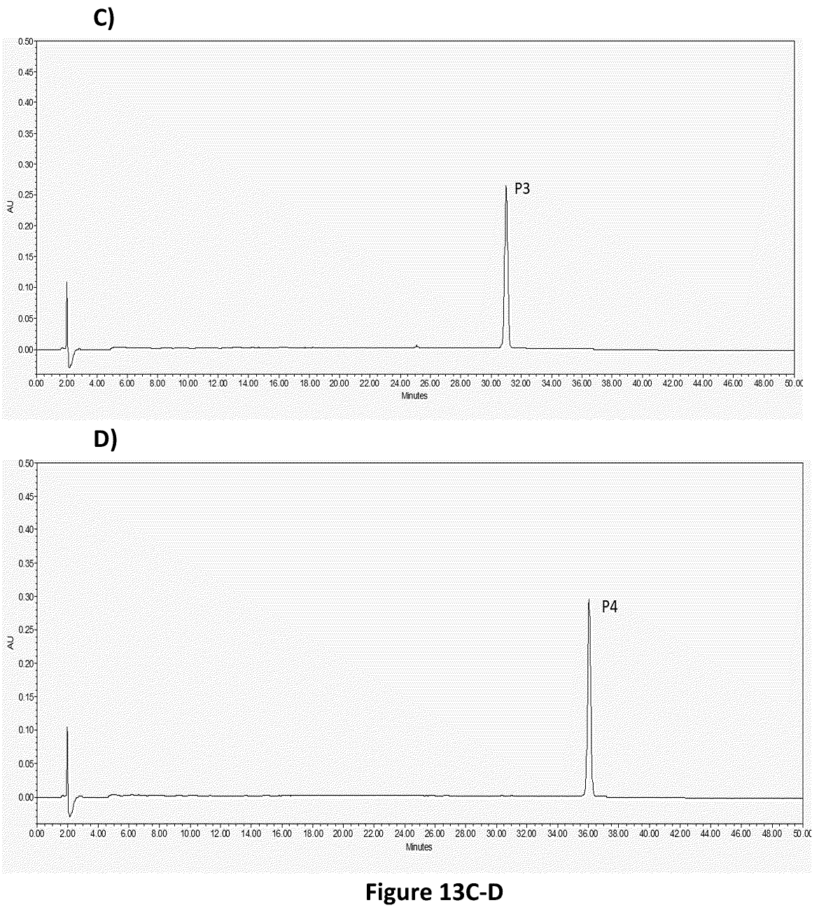

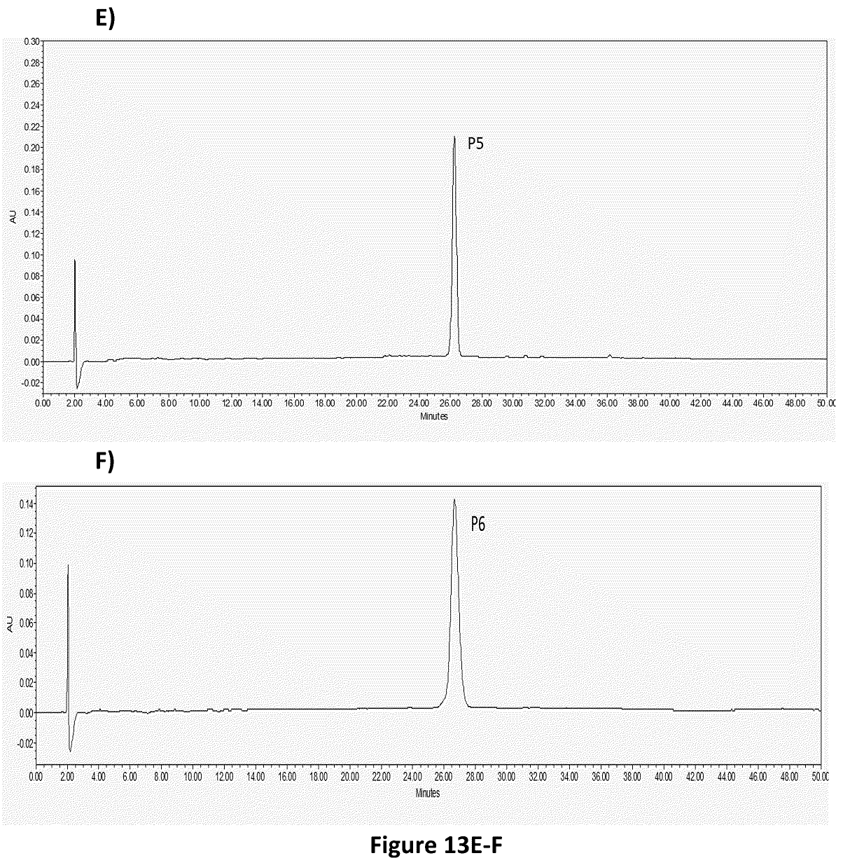

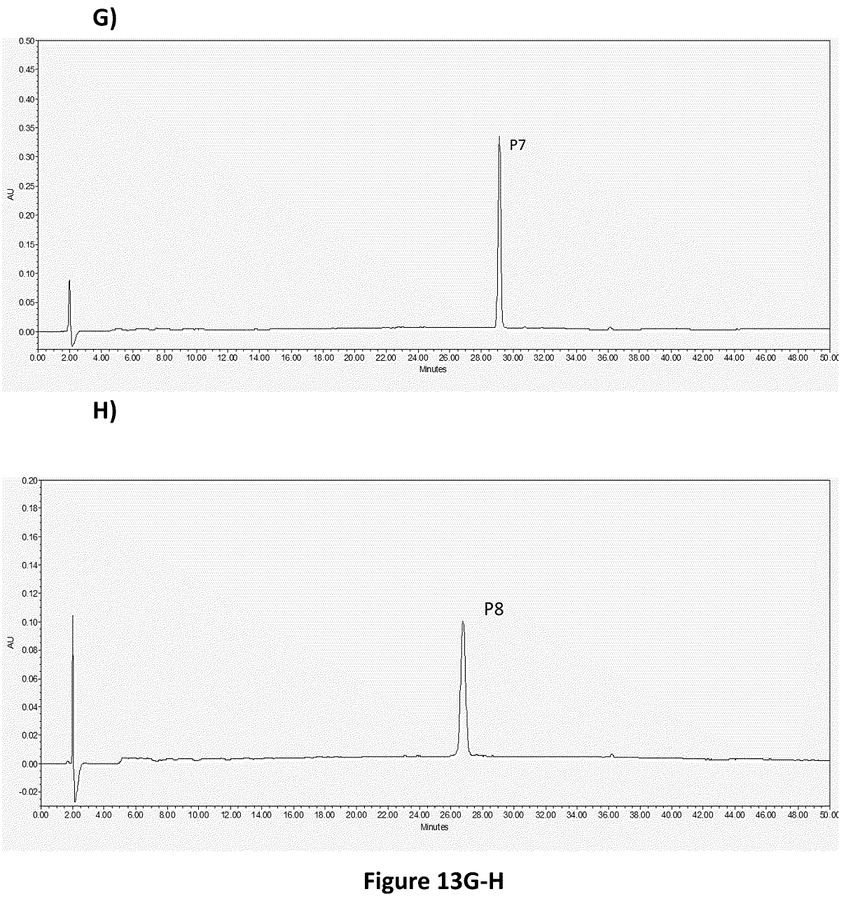

Rink amide resin (loading 0.4 mmoL/g) was used for the solid phase synthesis of γ-AA peptides and was purchased from GL Biochem. TentaGel resin (0.23 mmoL/g) was purchased from RAPP Polymere and used for preparation of OBTC library. All solvents and other chemical reagents used for building blocks synthesis were obtained from commercial suppliers and used without purification unless otherwise indicated. All the building blocks used for the library preparation was synthesized according to our previous research⁴⁸⁻⁴⁹. The cyclic peptides were purified and analyzed on a Waters Breeze 2 HPLC system installed with both an analytic module (1 mL/min) and a preparative module (16 mL/min) by employing a method using 5-100% linear gradient of solvent B (0.1% TFA in MeCN) in solvent A (0.1% TFA in H2O) over 45 min, followed by 100% solvent B over 5 min. All compounds are >95% pure by analytical HPLC. (FIG. 13). The molecular weight of each peptide was confirmed by high-resolution mass spectrometry obtained from Agilent 6220 using electrospray ionization time-of-flight (ESI-TOF). The MS/MS analysis was performed with an Applied Biosystems 4700 Proteomics Analyzer. HEK293 cells were from American Tissue Culture Collection (ATCC) and AlexaFluor488-conjugated FLAG Epitope tag monoclonal antibody was purchased from Sigma-Aldrich.

XL1 Blue cells were from Agilent Technologies (Santa Clara, CA, USA). BL21 (DE3) pLysS chemical competent cells were from Invitrogen for protein expression. pET-15b and pET-28a plasmids for protein expression were from Novagen (Madison, WI, USA). Uba1, UbcH7, HECT do-main and full-length protein of E6AP, UB and UbxD8 were expressed from pET28a-Uba1, pET15b-UbcH7, pET28a-E6AP HECT, pET28a-E6AP full-length, pET15b-UB and pET28a-UbxD8 plasmids, respectively. 0-catenin and HHR23A were expressed from pGEX-O-catenin and pGEX-HHR23A plasmid, respectively. HEK293T cells were from American Tissue Culture Collection (ATCC) and cultured in high-glucose Dulbecco's modified Eagles medium (DMEM) (Life Technologies, Carlsbad, CA, USA, 11965092) with 10% (v/v) Fetal bovine serum (FBS) (Life Technologies, 11965092). Anti-HHR23A antibody (sc-365669), anti-UB antibody (sc-8017), anti-UbxD8 anti-body (sc-374098), anti-E6AP antibody (sc-25509), anti-β-actin (sc-47778), Anti-β-catenin antibody (sc-65480), and anti-p53 antibody (sc-126) were from Santa Cruz Biotechnology. These antibodies were diluted between 500 and 1,000-fold to probe the Western blots. Anti-Flag M2 anti-body (F3165) was from Sigma-Aldrich and was diluted 2000-fold for Western blotting.

### Library Preparation

A detailed protocol was described in FIG. 4. Briefly, a split and pool method were used to prepare the γ-AA peptide-based OTBC library. Each bead was manipulated to have two layers: inner and outer layers. The γ-AA peptide was synthesized on the outer layer, in which the Fmoc protecting group of the γ-AA peptide building block was removed by 20% piperidine in DMF, and the exposed amino group reacted with the next γ-AA peptide building block using HOBt/DIC (6:6 equiv.) as the activation agents in DMF for 6 h. The Alloc protecting group in the γ-AA peptide building block was removed by 1% Pd(PPh₃)₄ and 10% Me₂NH—BH₃ in CH₂Cl₂, and the deprotected building block reacted with carboxylic acids in the presence of HOBt/DIC (6:6 equiv.) to introduce side chains. The decoding peptide was synthesized on the inner layer, in which the Dde group was removed using deprotection solution according to the previous report⁵⁰. The Dde protected amino acids were coupled onto the solid phase in DMF for 4 h in the presence of PyBop (6 equiv.) and NEM (6 equiv.).

### Library Screening

The TentaGel beads (200-250 μm, 13.67 g) were swelled in DMF for 1 h. After being washed with Tris buffer for three times, the beads were equilibrated in Tris buffer overnight at room temperature.

### Prescreening

Firstly, the TentaGel beads were incubated with blocking buffer (1% BSA in Tris buffer with a 1000× excess of E. coli lysate) for 1 h, then they were washed thoroughly with Tris buffer, and followed by the incubation with a AlexaFluor488-conjugated FLAG Epitope tag monoclonal antibody at a dilution of 1:500 for 2 h at room temperature. Then, the beads were washed with Tris buffer for three times and transferred into a 6-well plate to be observed under a fluorescence microscope. The beads emitting green fluorescence were picked up and excluded from formal screening. The rest of the beads were pooled into a peptide vessel. After being washed with Tris buffer, the beads were treated with 8 M guandine-HCl for 1 h to remove the bond protein at room temperature. Finally, the guandine-HCl was washed away with water and Tris buffer. The beads were then incubated in DMF for 1 h and followed by washing and equilibration in Tris buffer overnight.

### Screening

The beads were incubated with blocking buffer (1% BSA in Tris buffer with a 1000× excess of E. coli lysate) for 1 h at room temperature. After being washed with Tris buffer for three times, the beads were incubated with Flag-tagged E6AP domain at a concentration of 50 nM for 4 h with 1% BSA in Tris buffer and 1000× excess of E. coli lysate. After the thorough wash with Tris buffer, the library beads were incubated with 20 μL AlexaFluor488-conjugated FLAG Epitope tag monoclonal antibody in 10 mL Tris buffer at room temperature for 2 h. Then, the beads were washed with Tris buffer for three times and transferred into a 6-well plate to be observed under a fluorescence microscope. The positive beads were picked up based on the binding between the bead-anchored peptides and the HECT domain of E6AP, which have a Flag tag that was recognized by an anti-Flag antibody labeled with AlexaFluor 488 that would emit green fluorescence. The beads emitting green fluorescence were picked up as putative hits.

### Cleavage and Analysis

Each bead identified was transferred to a 1.5 mL microtube and denatured with 100 μL 8 M guandine-HCl for 1 h at room temperature, respectively. Then, the bead was rinsed with Tris buffer, water, DMF, and ACN for three times in sequence. At last, the bead was placed in ACN overnight and then the ACN was evaporated. The bead was incubated in the solution of ACN: glacial acetic acid: H₂O containing cyanogen bromide (CNBr) (v:v:v=5:4:1) at a concentration of 50 mg/mL overnight at room temperature. After the cleavage of the peptides from the bead, the solution was evaporated, and the cleaved peptide was dissolved in ACN: H₂O (4:1) and decoded by MALDI/MS.

### FITC-Labeled Peptides Preparation

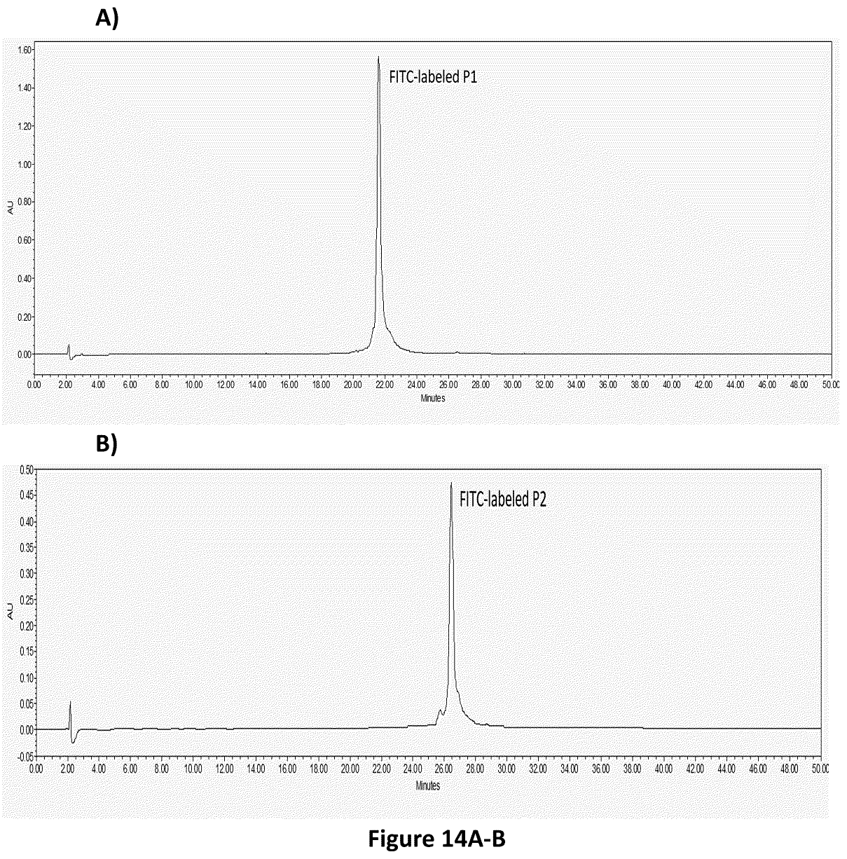

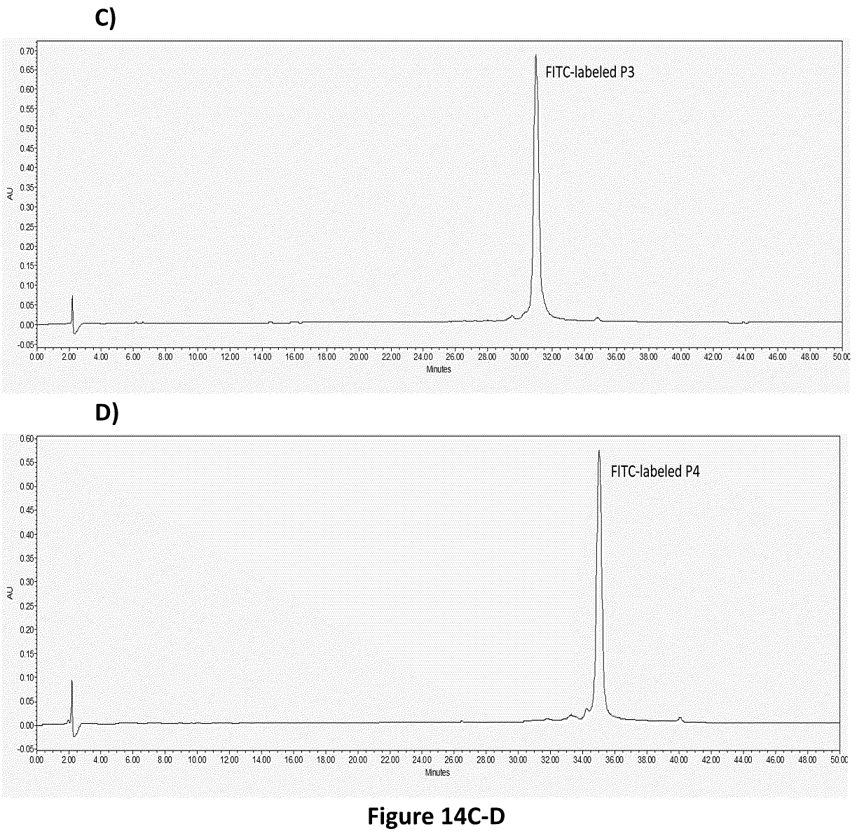

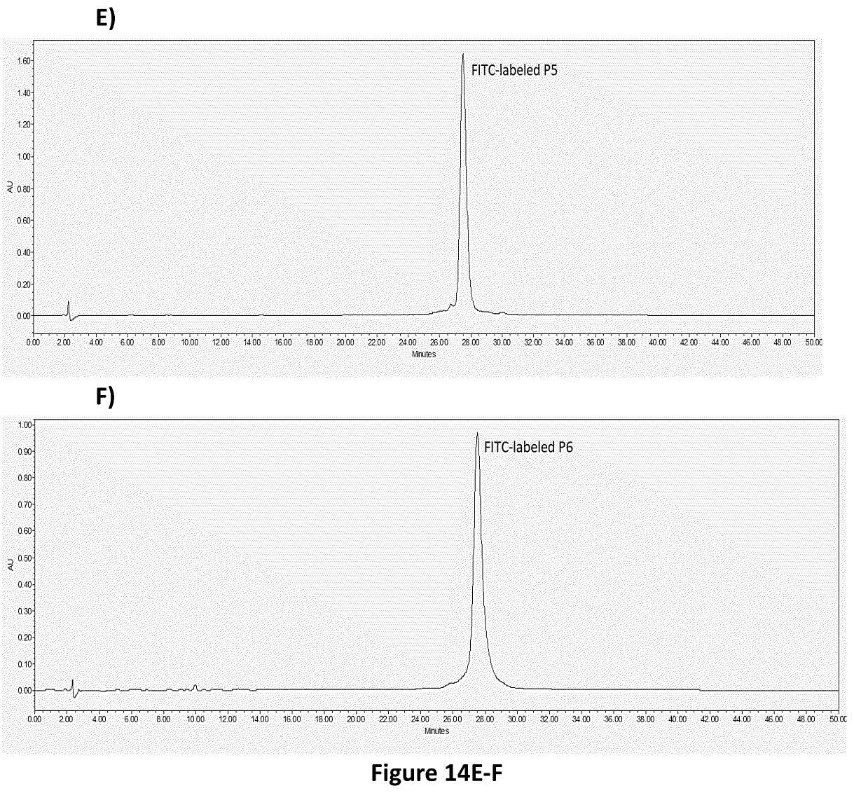

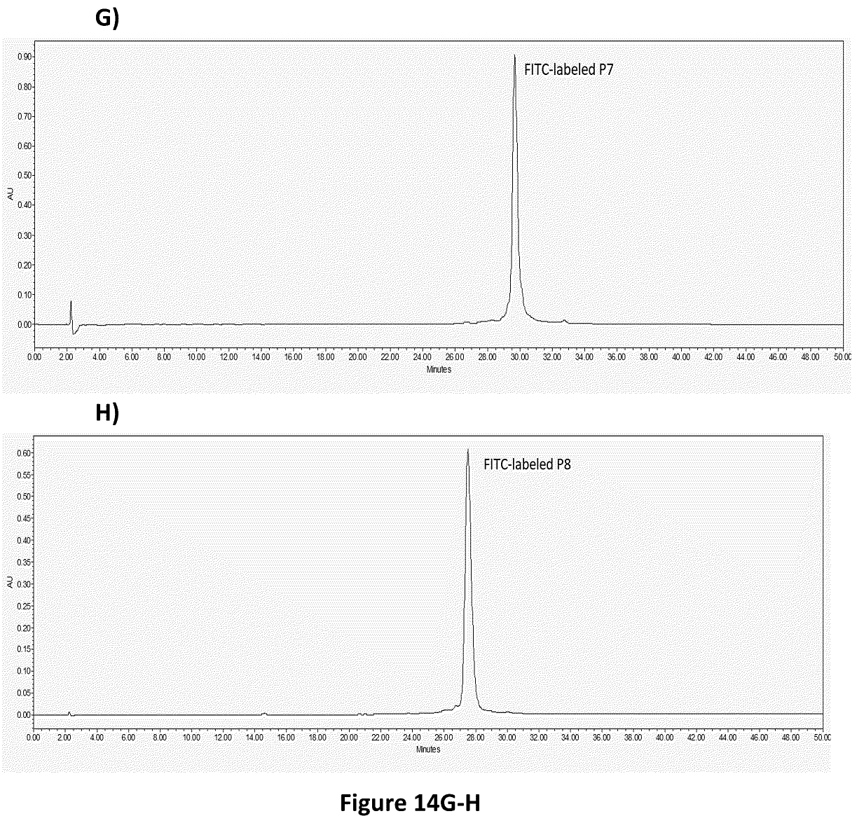

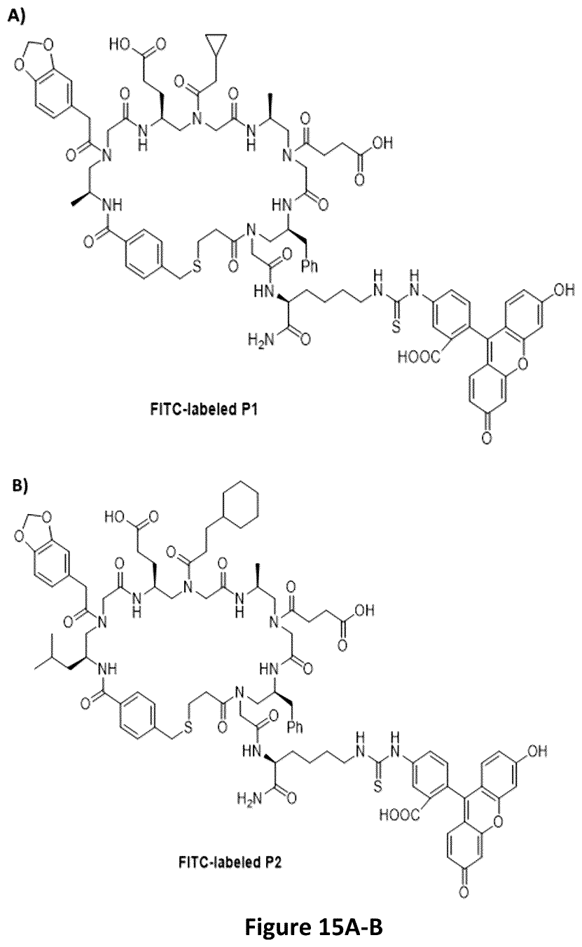

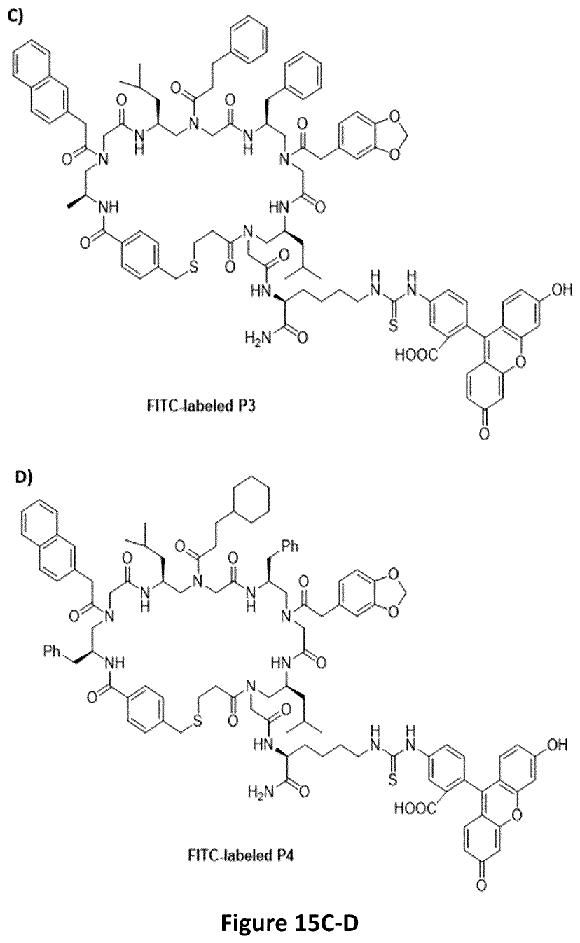

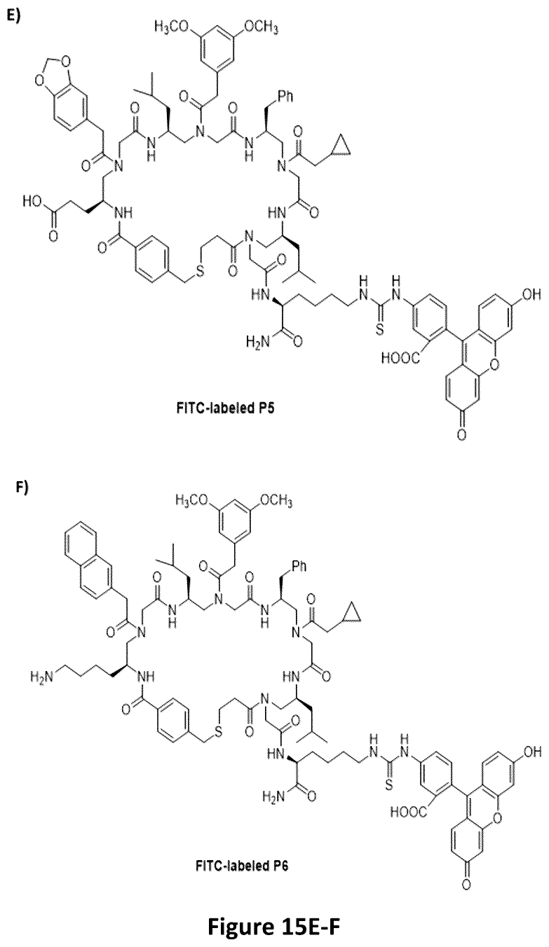

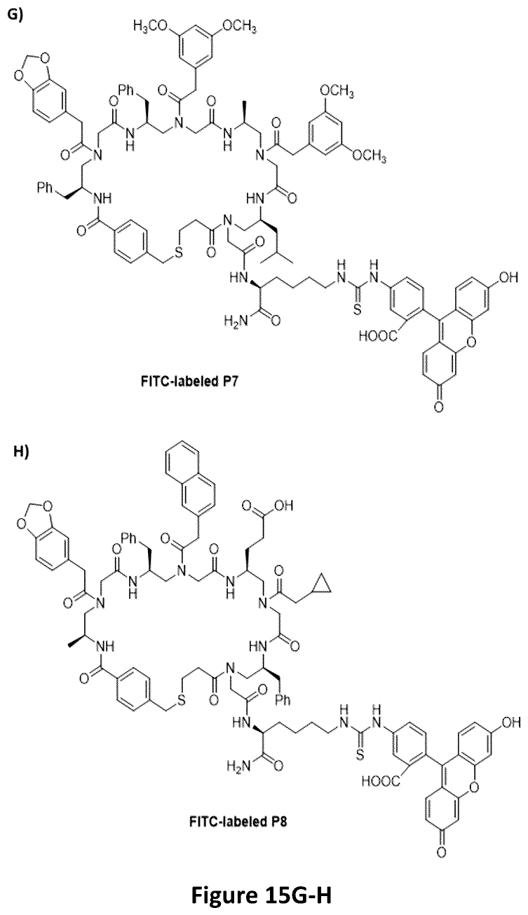

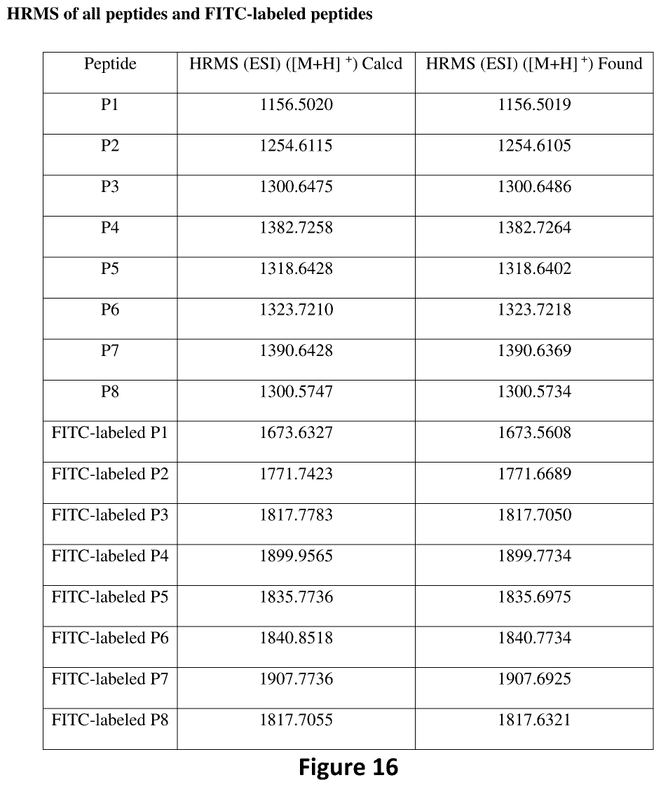

Fmoc-Lys(Dde)-OH was first attached to the Rink amide resin. Then, the Fmoc-protecting group was removed and followed by the coupling with desired building blocks. After cyclization, the Dde group was removed. FITC (2 equiv.) and DIPEA (10 equiv.) in DMF were added to the vessel and shaken overnight at room temperature. Then the FITC-labeled cyclic peptide was cleaved by 1:1 (v/v) DCM/TFA containing 2% triisopropylsilane. The crude was purified by the Waters HPLC. (FIG. 14). The detailed structures can be found in FIG. 15.

### Binding Affinity

The binding affinity (K_{d}) of the peptides were measured by fluorescence polarization. Briefly, a constant amount of the 100 nM FITC-labeled cyclic peptide was incubated with a serial dilution of E6AP HECT domain. The K_{d} values was calculated using the following equation, in which the Lₛₜ and x refer to the concentration of peptide and protein, respectively.

$$y = [ FP min + ( FP max - FP min ) ] \frac{( {K}_{d} + {L}_{st} + x ) - \sqrt{{( {K}_{d} + Lst + x )}^{2} - 4 {L}_{st} \times x}}{2 {L}_{st}} \qquad ( 1 )$$

Protein Expression from Recombinant pET Plasmids

Recombinant pET plasmids for the expression of Uba1, UbcH7, E6AP HECT domain, E6AP full-length, UB and UbxD8 were transformed into BL21 cells and cultured in 2λYT broth with antibiotics under 37° C. until the OD value of the media is within the range of 0.6-0.8. 1 mM IPTG was added to the cell culture to induce the expression and the cell culture was incubated overnight under 20° C. with agitation (220 rpm) before the cells were harvested by centrifugation (5,500 rpm, 4° C., 20 min). Cells were resuspended in 20 ml lysis buffer (50 mM NaH2PO4, 300 mM NaCl, 10 mM imidazole, pH 8.0) with the addition of 40 mg lysozyme and 1 mM PMSF and the mixture was incubated on ice for 30 min. The cell resuspension was sonicated on ice and the resulting cell lysate was centrifuged (10,000 rpm, 4° C., 30 min) and the supernatant was collected to bind with Ni-NTA beads overnight at 4° C. The protein was purified by a gravity-flow column with washes by 20 mL lysis buffer (50 mM NaH₂PO₄, 300 mM NaCl, 5 mM imidazole, pH 8.0) once and 20 mL wash buffer (50 mM NaH₂PO₄, 300 mM NaCl, 20 mM imidazole, pH 8.0) twice, followed by an elution with 5 mL elution buffer (50 mM NaH₂PO₄, 300 mM NaCl, 250 mM imidazole, pH 8.0). The eluted protein solution was further dialyzed in a dialysis buffer (50 mM Tris, 50 mM NaCl, 1 mM DTT, pH 8.0) and concentrated.

### In Vitro Assay to Measure the Effect of the Peptide Ligands on E6AP Self-Ubiquitination

All assays were set up in 50 μL reaction buffer supplemented with 50 mM Tris, 5 mM MgCl₂, 5 mM ATP and 1 mM DTT. 10 μM or 100 μM of each peptide was incubated with 0.5 μM wt Uba1, 0.5 μM wt UbcH7, 0.5 μM wt N-terminal Flag-tagged E6AP HECT domain or full-length protein at 37° C. for 30 min before 5 μM wt UB was added to start the UB-transfer reaction. The reactions were incubated for another 2 h at 37° C. and then quenched by boiling in the sample loading buffer of SDS-PAGE with DTT for 5 min and analyzed by SDS-PAGE and Western blot probed with anti-Flag antibody. In another experiment, P6 of gradient concentrations from 1 μM to 100 μM was added to the ubiquitination reaction mixture and the reactions proceeded similarly to assay substrate ubiquitination.

### In Vitro Assay to Measure the Effect of the Peptide Ligands on Substrate-Ubiquitination by E6AP

All assays were set up in 50 μL reaction buffer supplemented with 50 mM Tris, 5 mM MgCl2, 5 mM ATP and 1 mM DTT. 10 μM or 100 μM of each peptide was incubated with 0.5 μM wt Uba1, 0.5 μM wt UbcH7, 0.5 μM wt N-terminal Flag-tagged E6AP and 2 μM substrates (UbxD8, β-catenin or Rad23A) at 37° C. for 30 min before 5 μM wt UB was added to start the UB-transfer reaction. The reactions were incubated for another 2 h at 37° C. and then quenched by boiling in the sample loading buffer of SDS-PAGE with DTT for 5 min and analyzed by SDS-PAGE and Western blot probed with substrate-specific antibodies.

In another experiment, P6 of varying concentrations from 1 μM to 100 μM was added to the ubiquitination reaction mixture and the reactions proceeded similarly to assay substrate ubiquitination. The p53 ubiquitination assay was set up with the same concentrations of Uba1, UbcH7, and E6AP with the addition of 0.5 μM E6 protein. The enzymes and the peptide ligands were pre-incubated for 30 min before 5 μM wt UB was added to start the reaction. The reactions were incubated for another 2 h at 37° C. and then quenched by boiling in the sample loading buffer with DTT for 5 min and analyzed by SDS-PAGE. The Western blot was probed with an anti-p53 antibody.

Cellular assays to measure the stimulatory effects of P6 on ubiquitination of E6AP substrates

HEK293T cells were preincubated with P6 at 0, 25, and 50 μM for 14 h and with 0.5 μM MG132 for an additional 4 h. Cells were then washed twice with ice-cold PBS, pH 7.4, and 1 mL ice-cold RIPA buffer was added and incubated with the cells at 4° C. for 10 min. The cells were disrupted by repeated aspiration through a 21-gauge needle to induce cell lysis and the cell lysate was transferred to a 1.5 mL tube. The cell debris was pelleted by centrifugation at 13,000 r.p.m. for 20 min at 4° C. and the supernatant was transferred to a new tube and precleared by adding 1.0 μg of the appropriate control IgG (normal mouse or rabbit IgG corresponding to the host species of the primary antibody). 20 μL of sus-pended Protein A/G PLUS-agarose was added to the super-natant and the incubation was continued for 30 min at 4° C. After this, cell lysate containing 2 mg total protein was transferred to a new tube and 30 μL (i.e., 6 μg) primary antibody specific for UbxD8 or HHR23A was added. The incubation was continued for 1 h at 4° C. and 50 μL of resuspended Protein A/G PLUS-Agarose was added. The tubes were capped and incubated at 4° C. on a rocking plat-form overnight. The next day, the agarose beads were pelleted by centrifugation at 350×g for 5 min at 4° C. The beads were then washed 3 times, each time with 1.0 mL PBS. After the final wash, the beads were resuspended in 40 μL of 1× Laemmli buffer with β-mercaptoethanol. The samples were boiled for 5 min and analyzed by SDS-PAGE and Western blotting probed with an anti-UB antibody.

### E6AP Induced Protein Degradation in HEK293T Cells

Cycloheximide (CHX) chase assays were performed with HEK293T cells (5×106 cells) incubated with P6 at 0, 1, 5, and 50 μM for 14 hours. After the incubation, cells were treated with 100 μg/mL CHX to block de novo protein synthesis and harvested after variable incubation time with CHX. The levels of substrate proteins in the cell were assayed by immunoblotting with antibodies specific for UbxD8 and HHR23A. Protein levels were normalized to β-actin.

### Enzymatic Stability Study

0.01 mg/mL P6 was incubated with 0.1 mg/mL pronase in 100 mM ammonium bicarbonate buffer (pH 7.8) at 37° C. for 24 h. The reaction mixture was concentrated in a speed vacuum to remove the water and ammonium bicarbonate. The remains were dissolved in 100 μL H2O/CH3CN and analyzed by a Waters analytical HPLC system with a 1 mL/min flow rate and 5-100% linear gradient of solvent B (0.1% TFA in acetonitrile) in A (0.1% TFA in water) over the duration of 50 min. The UV detector was set to 215 nm.

### Serum Stability Assay

The serum stabilities of peptides were determined in 25% (v/v) aqueous pooled serum from human male AB plasma (Sigma-Aldrich, Milan, Italy). 1 mg P6 was dissolved in 50 μL CH3CN/H2O (70:30, v/v) and then diluted in serum and incubated at 37° C. for 24 h. Then, 100 μL of solution was added to 100 μL of CH3CN on ice for 15 min and was centrifuged at 4° C. for 10 min. The supernatant was then analyzed by a Waters analytical HPLC system with a 1 mL/min flow rate and 5-100% linear gradient of solvent B (0.1% TFA in acetonitrile) in A (0.1% TFA in water) over the duration of 50 min. The UV detector was set to 215 nm.

### CONCLUSION

γ-AA peptides have exhibited promising potentials for biological application and drug discovery. Due to their propensity to form helical structures akin to α-helices of proteins³⁸⁻⁴¹, -AA peptides can mimic host-defense peptides³⁶⁻³⁷ and modulate disease-related protein-protein interactions such as p53/MDM2, β-catenin/BCL9, and GLP-1/GLP-1R in vitro and in vivo⁴²⁻⁴⁵. Additionally, owing to the convenience for incorporating unnatural functionalities to their scaffolds with the modular synthesis protocol, γ-AA peptides are ideal for generating diverse libraries to screen for ligands of biological targets²⁹⁻³¹.

The inventors conducted an affinity-based screen to identify γ-AA peptides that can bind to the HECT domain of E6AP with submicromolar binding affinity. Among the ligands deduced from the library, peptide P6 stood out as a potent activator of E6AP. It was found that not only can P6 stimulate self-ubiquitination of E6AP and E6AP-catalyzed substrate ubiquitination in reconstituted reactions in vitro, but it can also enhance the ubiquitination of E6AP substrates in the cell and accelerate their degradation by the proteasome. Such an E3 ligand that can stimulate the UB ligase activity of an E3 is unique in that it can occupy a distinctive therapeutic landscape from the many E3 inhibitors that are being moved through the drug discovery pipeline⁴. Both an overactive E3 due to dysregulation or an underperforming E3 due to genetic mutations can be causative of diseases. The discovery of E3-stimulating ligands, such as P6 for E6AP, may boost the activity of mutated E3s in the cell to restore their normal functions.

The inventors also found the P6 peptide would not further activate E6AP when there was viral E6 protein from HPV present. Investigation into whether P6 and E6 share the same binding mode with E6AP is warranted.

Previously phage-displayed libraries of UB variants (UbVs) have been selected for binding to the HECT domains of various E3s, and some of the UbVs from the selection were found to activate HECT E3s Nedd4 and Nedd4L⁴⁶. Peptide and small molecule ligands of E3 would be better leads for drug development so N-methyl-cyclic peptides were identified for binding to the HECT domain of E6AP and inhibiting its UB ligase activity¹⁵. Yet the activity of the N-methyl-peptide in the cell was not characterized. In another report, small molecule ligands of flavin derivatives were found to activate E6AP to enhance its ubiquitination of substrate proteins²⁵. Interestingly, the flavin-like ligands would induce a conformational change of E6AP close to the effect of HPV E6 on E6AP²⁵. The effects of the flavin-like ligands on E6AP activity in the cell were not characterized. However, testing if the P6 γ-AA peptide would target the same binding site of the flavin ligand in E6AP and if P6 would induce a similar conformational change in E6AP to activate its ligase activity could be warranted.

The advantage of using the γ-AA peptide scaffold for developing ligands to affect E3 activities in the cell was presented. The enzymatic cascades of UB transfer rely on sophisticated protein-protein interactions to deliver UB to the substrate proteins. Peptides and their structural mimics such as γ-AA peptides would have a better chance than small molecules to manipulate protein-protein interactions to inhibit or activate enzymes of the UB-transfer cascades. Indeed, recent success in designing stapled peptides to perturb E1-E2 interactions demonstrates the potential of peptide ligands as a privileged scaffold to target protein ubiquitinating enzymes⁴⁷. The remarkable stability of the γ-AA peptides at physiological conditions would provide another advantage for developing them to activate or inhibit protein ubiquitination pathways.

### Example 2—Stimulation of E6AP in Angelman's Syndrome Patient (Prophetic)

Assays are conducted on P6 and other peptide ligands from the screen to determine if they may activate mutated E6AP that are causative for neurodegenerative diseases such as Angelman syndrome. The screen of the γ-AA peptide library is also repeated with the mutated E6AP to identify additional ligands that can restore the activity of the E3 implicated in Angelman syndrome. The inventors find that P6 is able to also activate mutated E6AP.

A human child presents with severe developmental delay that becomes apparent around the age of 9 months. The child later presents with absent speech, hypotonia, ataxia and microcephaly. The child moves with a jerky, puppet like gait and displays an unusually happy demeanor that is accompanied by laughing spells. The child has dysmorphic facial features characterized by a prominent chin, an unusually wide smile and deep-set eyes. The child diagnoses with Angelman's Syndrome.

The child is treated with a therapeutically effective amount of compound P6 over a period of time. Improvement is seen in the symptoms after treatment with a decrease in seizures, increased nuscle tone, increased coordination of muscle movement and improvement in speech.

### REFERENCES

(1) Finley, D., Ciechanover, A., Varshavsky, A. Ubiquitin as a central cellular regulator. Cell. 2004, 116, S29-32.
(2) Schwartz, A. L., Ciechanover, A. Targeting proteins for destruction by the ubiquitin system: implications for human pathobiology. Annu. Rev. Pharmacol. Toxicol. 2009, 49, 73-96.
(3) Bedford, L., Lowe, J., Dick, L. R., Mayer, R. J., Brownell, J. E. Ubiquitin-like protein conjugation and the ubiquitin-proteasome system as drug targets. Nat. Rev. Drug Discov. 2011, 10, 29-46.
(4) Wertz, I. E., Wang, X. From Discovery to Bedside: Targeting the ubiquitin system. Cell. Chem. Biol. 2019, 26, 156-177.
(5) Gerry, C. J., Schreiber, S. L. Unifying principles of bifunctional, proximity-inducing small molecules. Nat. Chem. Biol. 2020, 16, 369-378.
(6) Lai, A. C., Crews, C. M. Induced protein degradation: an emerging drug discovery paradigm. Nat. Rev. Drug Discov. 2017, 16, 101-114.
(7) Wang, Y., Liu, X., Zhou, L., Duong, D., Bhuripanyo, K., Zhao, B., Zhou, H., Liu, R., Bi, Y., Kiyokawa, H., Yin, J. Identifying the ubiquitination targets of E6AP by orthogonal ubiquitin transfer. Nat. Commun. 2017, 8, 2232.
(8) Huang, L., Kinnucan, E., Wang, G., Beaudenon, S., Howley, P. M., Huibregtse, J. M., Pavletich, N. P. Structure of an E6AP-UbcH7 complex: insights into ubiquitination by the E2-E3 enzyme cascade. Science. 1999, 286, 1321-1326.
(9) Guntas, G., Purbeck, C., Kuhlman, B. Engineering a protein-protein interface using a computationally designed library. Proc. Natl. Acad. Sci. USA 2010, 107, 19296-19301.
(10) Ronchi, V. P., Kim, E. D., Summa, C. M., Klein, J. M., Haas, A. L. In silico modeling of the cryptic E2 approximately ubiquitin-binding site of E6-associated protein (E6AP)/UBE3A reveals the mechanism of polyubiquitin chain assembly. J. Biol. Chem. 2017, 292, 18006-18023.
(11) Mortensen, F., Schneider, D., Barbic, T., Sladewska-Marquardt, A., Kuhnle, S., Marx, A., Scheffner, M. Role of ubiquitin and the HPV E6 oncoprotein in E6AP-mediated ubiquitination. Proc. Natl. Acad. Sci. USA 2015, 112, 9872-9877.
(12) Huibregtse, J. M., Scheffner, M., Howley, P. M. A cellular protein mediates association of p53 with the E6 oncoprotein of human papillomavirus types 16 or 18. Embo. J. 1991, 10, 4129-4135.
(13) Scheffner, M., Werness, B. A., Huibregtse, J. M., Levine, A. J., Howley, P. M. The E6 oncoprotein encoded by human papillomavirus types 16 and 18 promotes the degradation of p53. Cell. 1990, 63, 1129-1136.
(14) Scheffner, M., Huibregtse, J. M., Vierstra, R. D., Howley, P. M. The HPV-16 E6 and E6-AP complex functions as a ubiquitin-protein ligase in the ubiquitination of p53. Cell. 1993, 75, 495-505.
(15) Yamagishi, Y., Shoji, I., Miyagawa, S., Kawakami, T., Katoh, T., Goto, Y., Suga, H. Natural product-like macrocyclic N-methyl-peptide inhibitors against a ubiquitin ligase uncovered from a ribosome-expressed de novo library. Chem. Biol. 2011, 18, 1562-1570.
(16) Malecka, K. A., Fera, D., Schultz, D. C., Hodawadekar, S., Reichman, M., Donover, P. S., Murphy, M. E., Marmorstein, R. Identification and characterization of small molecule human papillomavirus E6 inhibitors. ACS Chem. Biol. 2014, 9, 1603-1612.
(17) Sterlinko Grm, H., Weber, M., Elston, R., McIntosh, P., Griffin, H., Banks, L., Doorbar, J. Inhibition of E6-induced degradation of its cellular substrates by novel blocking peptides. J. Mol. Biol. 2004, 335, 971-985.
(18) Cherry, J. J., Rietz, A., Malinkevich, A., Liu, Y., Xie, M., Bartolowits, M., Davisson, V. J., Baleja, J. D., Androphy, E. J. Structure based identification and characterization of flavonoids that disrupt human papillomavirus-16 E6 function. PLoS One. 2013, 8, e84506.
(19) Zanier, K., Stutz, C., Kintscher, S., Reinz, E., Sehr, P., Bulkescher, J., Hoppe-Seyler, K., Trave, G., Hoppe-Seyler, F. The E6AP binding pocket of the HPV16 E6 oncoprotein provides a docking site for a small inhibitory peptide unrelated to E6AP, indicating druggability of E6. PLoS One. 2014, 9, e112514.
(20) George, A. J., Hoffiz, Y. C., Charles, A. J., Zhu, Y., Mabb, A. M. A comprehensive atlas of E3 ubiquitin ligase mutations in neurological disorders. Front. Genet. 2018, 9, 29.
(21) Mabb, A. M., Judson, M. C., Zylka, M. J., Philpot, B. D. Angelman syndrome: insights into genomic imprinting and neurodevelopmental phenotypes. Trends Neurosci. 2011, 34, 293-303.
(22) Wolter, J. M., Mao, H., Fragola, G., Simon, J. M., Krantz, J. L., Bazick, H. O., Oztemiz, B., Stein, J. L., Zylka, M. J. Cas9 gene therapy for angelman syndrome traps Ube3a-ATS long non-coding RNA. Nature. 2020, 587, 281-284.
(23) Meng, L., Ward, A. J., Chun, S., Bennett, C. F., Beaudet, A. L., Rigo, F. Towards a therapy for angelman syndrome by targeting a long non-coding RNA. Nature. 2015, 518, 409-412.
(24) Huang, H. S., Allen, J. A., Mabb, A. M., King, I. F., Miriyala, J., Taylor-Blake, B., Sciaky, N., Dutton, J. W., Jr., Lee, H. M., Chen, X., Jin, J., Bridges, A. S., Zylka, M. J., Roth, B. L., Philpot, B. D. Topoisomerase inhibitors unsilence the dormant allele of Ube3a in neurons. Nature. 2011, 481, 185-189.
(25) Offensperger, F., Muller, F., Jansen, J., Hammler, D., Gotz, K. H., Marx, A., Sirois, C. L., Chamberlain, S. J., Stengel, F., Scheffner, M. Identification of small-molecule activators of the ubiquitin ligase E6AP/UBE3A and angelman syndrome-derived E6AP/UBE3A variants. Cell. Chem. Biol. 2020, 7, 1510-1520.
(26) Sang, P., Shi, Y., Huang, B., Xue, S., Odom, T., Cai, J. Sulfono-γ-AApeptides as helical mimetics: crystal structures and applications. Acc. Chem. Res. 2020, 53, 2425-2442.
(27) Rotin, D., Kumar, S. Physiological functions of the HECT family of ubiquitin ligases. Nat. Rev. Mol. Cell. Biol. 2009, 10, 398-409.
(28) Fajner, V., Maspero, E., Polo, S. Targeting HECT-type E3 ligases—insights from catalysis, regulation and inhibitors. FEBS Lett. 2017, 591, 2636-2647.
(29) Shi, Y., Challa, S., Sang, P., She, F., Li, C., Gray, G. M., Nimmagadda, A., Teng, P., Wang, Y., Vaart, A., Li, Q., Cai, J. One-bead-two-compound thioether bridged macrocyclic γ-AApeptide screening library against EphA2. J. Med. Chem. 2017, 60, 9290-9298.
(30) Yan, H., Zhou, M., Bhattarai, U., Song, Y., Zheng, M., Cai, J. Cyclic peptidomimetics as inhibitor for miR-155 biogenesis. Mol. Pharmaceutics. 2019, 16, 914-920.
(31) Shi, Y., Parag, S., Patel, R., Liu, A., Murr, M., Cai, J., Patel, N. A. Stabilization of lncRNA GAS5 by a small molecule and its implications in diabetic adipocytes. Cell. Chem. Biol. 2019, 26, 319-330.
(32) Kuslansky, Y., Sominsky, S., Jackman, A., Gamell, C., Monahan, B. J., Haupt, Y., Rosin-Arbesfeld, R., Sherman, L. Ubiquitin ligase E6AP mediates nonproteolytic polyubiquitylation of beta-catenin independent of the E6 oncoprotein. J. Gen. Virol. 2016, 97, 3313-3330.
(33) Yi, J. J., Paranjape, S. R., Walker, M. P., Choudhury, R., Wolter, J. M., Fragola, G., Emanuele, M. J., Major, M. B., Zylka, M. J. The autism-linked UBE3A T485A mutant E3 ubiquitin ligase activates the Wnt/beta-catenin pathway by inhibiting the proteasome. J. Biol. Chem. 2017, 292, 12503-12515.
(34) Kumar, S., Talis, A. L., Howley, P. M. Identification of HHR23A as a substrate for E6-associated protein-mediated ubiquitination. J. Biol. Chem. 1999, 274, 18785-18792.
(35) Vande Pol, S. B., Klingelhutz, A. J. Papillomavirus E6 oncoproteins. Virology. 2013, 445, 115-137.
(36) Wang, M., Gao, R., Zheng., M., Sang, P., Li, C., Zhang, E., Li, Q., Cai, J. Development of bis-cyclic imidazolidine-4-one derivatives as potent antibacterial agents. J. Med. Chem. 2020, 63, 15591-15602.
(37) Wang, M., Feng, X., Gao, R., Sang, P., Pan, X., Wei, L., Lu, C., Wu, C., Cai, J. Modular design of membrane-active antibiotics: From macromolecular antimicrobials to small scorpionlike peptidomimetics. J. Med. Chem. 2021, 64, 9894-9905.
(38) She, F., Teng, P., Tejada, A. P., Wang, M., Ma, N., Odom, T., Zhou, M., Gjonaj, E., Wojtas, L., Vaart, A. V. D., Cai, J. De novo left-handed synthetic peptidomimetic foldamers. Angew. Chem. Int. Ed. 2018, 57, 9916-9920.
(39) Teng, P., Gray, G. M., Zheng, M. Singh, S., Li, X., Wojtas, L., Vaart, A. V. D., Cai, J. Orthogonal halogen-bonding-driveb 3D supramolecular assembly of right-handed synthetic helical peptides. Angew. Chem. Int. Ed. 2019, 58, 7778-7782.
(40) Shi, Y., Yin, G., Yan, Z., Sang, P., Wang, M., Brzozowski, R., Eswara, P., Wojtas, L., Zheng, Y., Li, X., Cai, J. Helical sulfono-γ-AApeptides with aggregaton-induced emission and circularly polarized luminescence. J. Am. Chem. Soc. 2019, 141, 12697-12706.
(41) Teng, P., Zheng, M., Cerrato, D. C., Shi, Y., Zhou, M., Xue, S., Jiang, W., Wojtas, L., Ming, L., Hu, Y., Cai, J. The folding propensity of α/sulfono-γ-AA peptidic foldamers with both left- and right-handedness. Commun. Chem. 2021, 4, 58.
(42) Sang, P., Zhang, M., Shi, Y., Li, C., Adbulkadir, S., Ji, H., Cai, J. Inhibition of (3-catenin/B cell lymphoma 9 protein-protein interaction using α-helix-mimicking sufono-γ-AApeptide inhibitors. Proc. Natl. Acad. Sci. USA 2019, 116, 10757-10762.
(43) Sang, P., Zhou, Z., Shi, Y., Lee, C., Amso, Z., Huang, D., Odom, T., Nguyen-Tran, V. T. B., Shen, W., Cai, J. The activity of sulfono-γ-AApeptide helical foldamers that mimic GLP-1. Science Advances. 2020, 6, eaaz4988.
(44) Sang, P., Shi, Y., Lu. J., Chen, L., Yang, L., Borcherds, W., Abdulkadir, S. Li, Q., Daughdrill, G., Chen, J., Cai, J. α-Helix-mimicking sulfono-γ-AApeptide inhibitors for p53-MDM2/MDMX protein-protein interactions. J. Med. Chem. 2020, 63, 975-986.
(45) Shi, Y., Sang, P., Lu, J., Higbee, P., Chen, L., Yang, L., Odom, T., Daughdrill, G., Chen, J., Cai, J. Rational design of eight-handed heterogeneous peptidomimetics as inhibitors of protein-protein interactions. J. Med. Chem. 2020, 63, 13187-13196.
(46) Zhang, W., Wu, K. P., Sartori, M. A., Kamadurai, H. B., Ordureau, A., Jiang, C., Mercredi, P. Y., Murchie, R., Hu, J., Persaud, A., Mukherjee, M., Li, N., Doye, A., Walker, J. R., Sheng, Y., Hao, Z., Li, Y., Brown, K. R., Lemichez, E., Chen, J., Tong, Y., Harper, J. W., Moffat, J., Rotin, D., Schulman, B. A., Sidhu, S. S. System-wide modulation of HECT E3 ligases with selective ubiquitin variant probes. Mol. Cell. 2016, 62, 121-36.
(47) Cathcart, A. M., Bird, G. H., Wales, T. E., Herce, H. D., Harvey, E. P., Hauseman, Z. J., Newman, C. E., Adhikary, U., Prew, M. S., Oo, T., Lee, S., Engen, J. R., Walensky, L. D. Targeting a helix-in-groove interaction between E1 and E2 blocks ubiquitin transfer. Nat. Chem. Biol. 2020, 16, 1218-1226.
(48) Wu, H., Teng, P., Cai, J. Rapid access to multiple classes of peptidomimetics from common γ-AApeptide building blocks. Eur. J. Org. 2014, 8, 1760-1765.
(49) Niu, Y., Bai, G., Wu, H., Wang, R., Qiao, Q., Padhee, S., Buzzeo, R., Cao, H., Cai, J. Cellular translocation of a γ-AApeptide mimetic of Tat peptide. Mol. Pharmaceutics. 2012, 9, 1529-1534.
(50) Díaz-Mochón, J. J., Bialy, L., Bradley, M. Full orthogonality between Dde and Fmoc: The direct synthesis of PNA-Peptide conjugates. Org. Lett. 2004, 6, 1127-1129.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A compound of Formula (I-VIII) comprising:

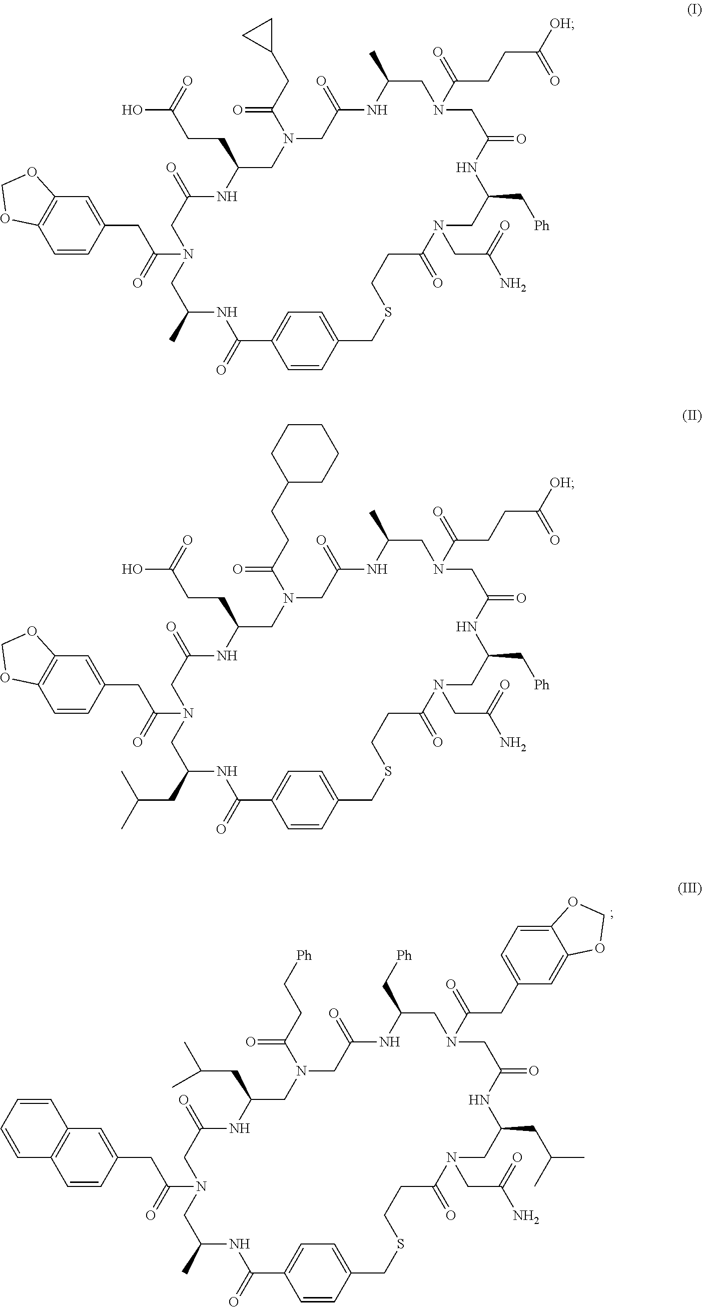

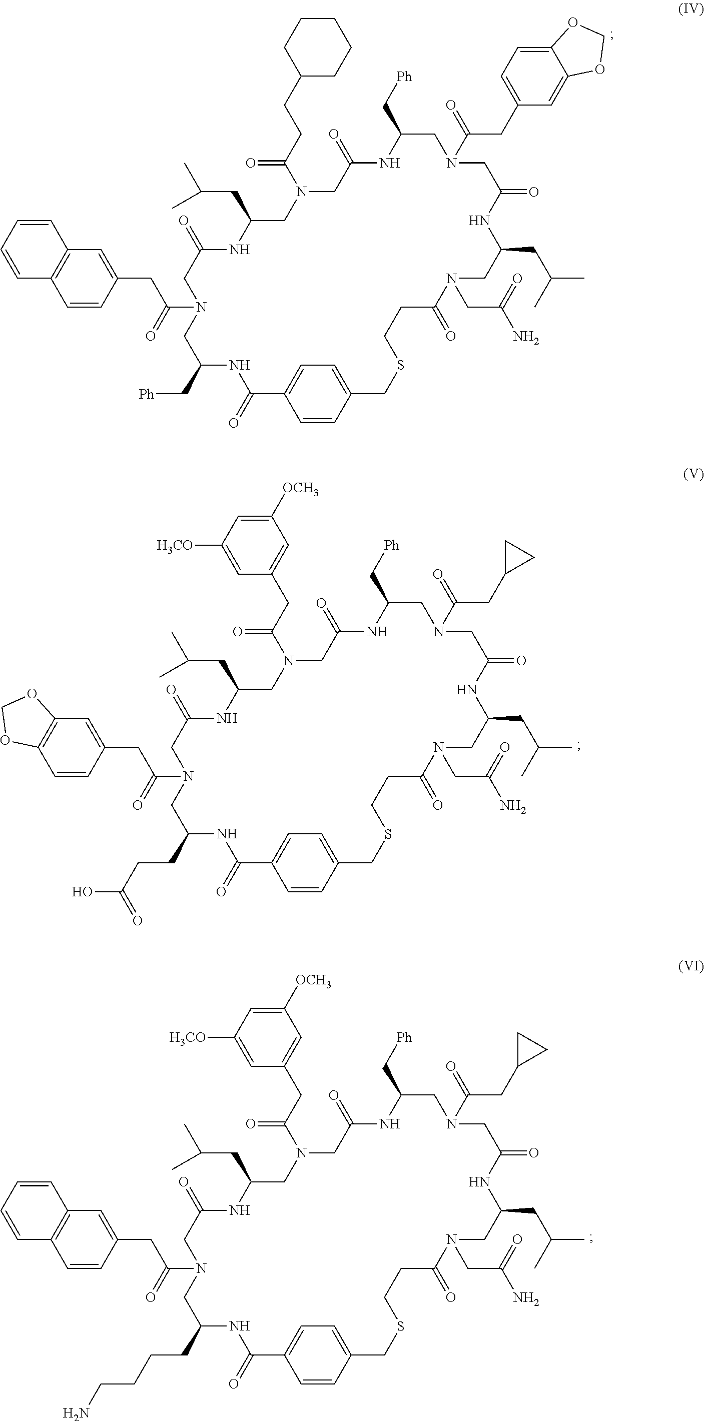

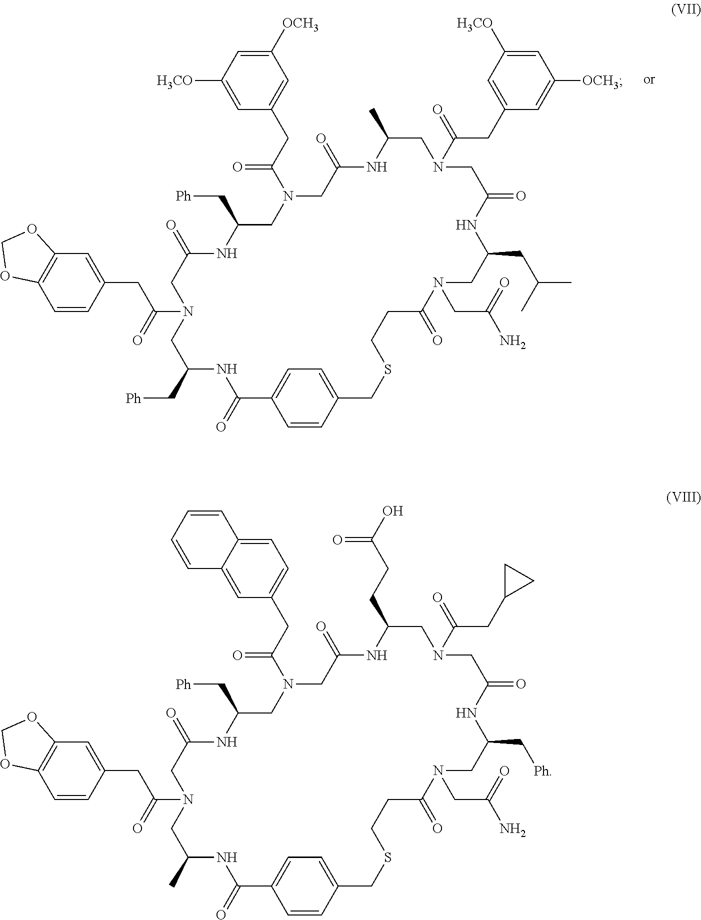 [SMILES: COc1cc(CC(=O)N2CC(=O)N[C@@H](C)CN(C(=O)Cc3cc(OC)cc(OC)c3)CC(=O)N[C@@H](CC(C)C)CN(CC(N)=O)C(=O)CCSCc3ccc(cc3)C(=O)N[C@@H](Cc3ccccc3)CN(C(=O)Cc3ccc4c(c3)OCO4)CC(=O)N[C@@H](Cc3ccccc3)C2)cc(OC)c1 ; "or" ; C[C@H]1CN(C(=O)Cc2ccc3c(c2)OCO3)CC(=O)N[C@@H](Cc2ccccc2)CN(C(=O)Cc2ccc3ccccc3c2)CC(=O)N[C@@H](CCC(=O)O)CN(C(=O)CC2CC2)CC(=O)N[C@@H](Cc2ccccc2)CN(CC(N)=O)C(=O)CCSCc2ccc(cc2)C(=O)N1]

2. A method of stimulating E3 ubiquitin ligase activity in a cell comprising:
contacting the cell with a therapeutically effective amount of at least one γ-substituted-N-acylated-N-aminoethyl amino acids (γ-AA peptide) compound;
wherein the γ-AA peptide stimulates the E3 ubiquitin ligase activity in the cell.

3. The method of claim 2, wherein the E3 is E6 associated protein (E6AP).

4. The method of claim 3, wherein the γ-AA peptide is cyclic.

5. The method of claim 2, wherein the γ-AA peptide is formed from at least one γ-AA building block.

6. The method of claim 5, wherein the at least one γ-AA building block of Formula (IX) comprises: 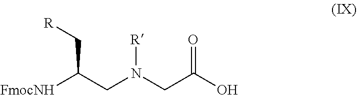
wherein R is selected from the group consisting of H, 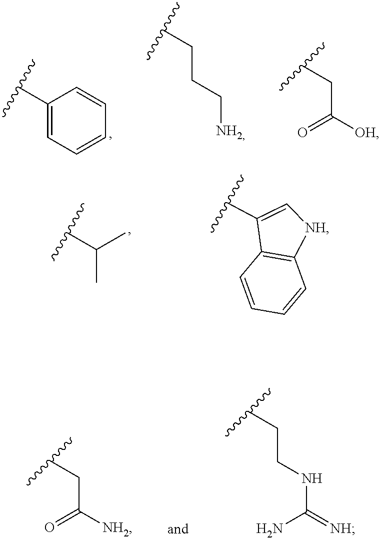
wherein R′^{μ} is selected from the group consisting of

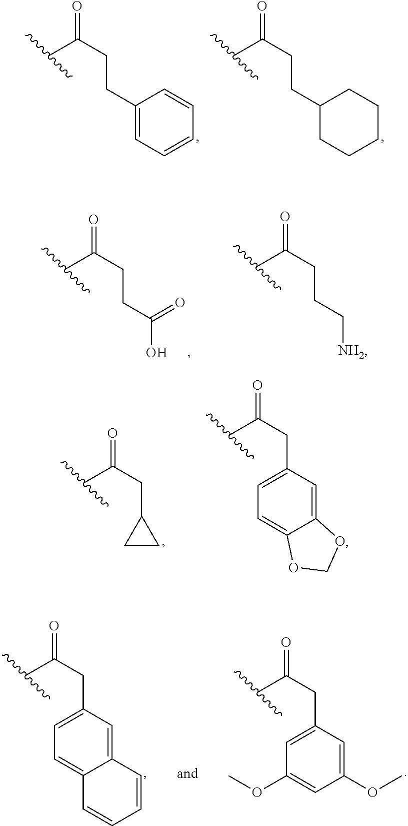

7. The method of claim 6, wherein the γ-AA peptide is formed from four of the 7-AA peptide building blocks.

8. The method of claim 3, wherein the γ-AA peptide is selected from the group consisting of Formula (I), Formula (II), Formula (III), Formula (IV), Formula (V), Formula (VI), Formula (VII) and Formula (VIII) of claim 1.

9. The method of claim 8, wherein the γ-AA peptide is the compound of Formula (VI).

10. A method of inducing E6 associated protein (E6AP) substrate protein degradation in a cell comprising:
contacting the cell with a therapeutically effective amount of at least one cyclic 7-substituted-N-acylated-N-aminoethyl amino acids (γ-AA peptide) compound;
wherein the γ-AA peptide induces E6AP substrate protein degradation by proteosome in the cell.

11. The method of claim 10, wherein the γ-AA peptide enhances ubiquitination of E6 associated protein (E6AP) substrate proteins in the cell.

12. The method of claim 10, wherein the γ-AA peptide is formed from four γ-AA peptide building blocks.

13. The method of claim 12, wherein each of the four γ-AA building blocks of Formula (IX) comprises: 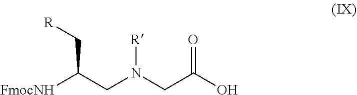
wherein R is selected from the group consisting of H, 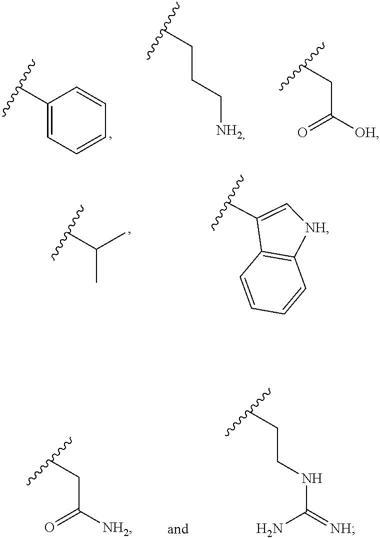
wherein R′ is selected from the group consisting of

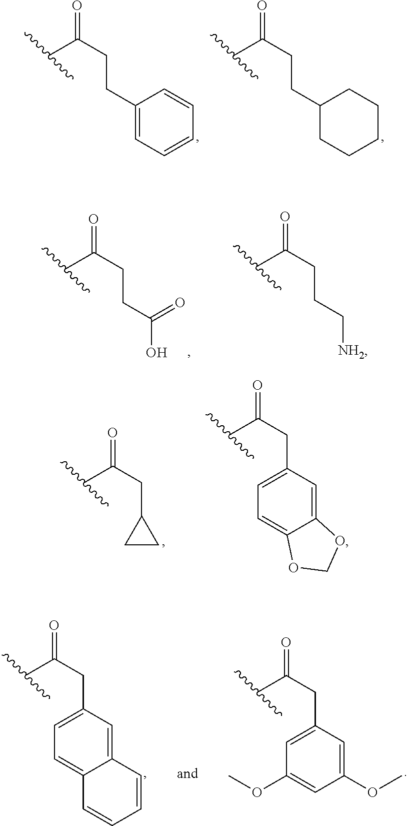

14. The method of claim 10, wherein the γ-AA peptide is selected from the group consisting of Formula (I), Formula (II), Formula (III), Formula (IV), Formula (V), Formula (VI), Formula (VII) and Formula (VIII) of claim 1.

15. The method of claim 14, wherein the γ-AA peptide is the compound of Formula (VI).
